(19) United States
(12) Patent Application Publication
(10) Pub. No.: US 2024/0247224 A1
(43) Pub. Date: Jul. 25, 2024

(54) SYSTEM AND METHOD FOR RAPID SUSCEPTIBILITY TESTING

(71) Applicants: Andrew Patrick Isakson, Cornelius, NC (US); Mitchell David Newton, Kansas City, MO (US); Sachidevi Puttaswamy, Overland Park, KS (US); Roy Raymond Swiger, Indialantic, FL (US)

(72) Inventors: Andrew Patrick Isakson, Cornelius, NC (US); Mitchell David Newton, Kansas City, MO (US); Sachidevi Puttaswamy, Overland Park, KS (US); Roy Raymond Swiger, Indialantic, FL (US)

(21) Appl. No.: 18/417,703

(22) Filed: Jan. 19, 2024

Related U.S. Application Data
(60) Provisional application No. 63/480,869, filed on Jan. 20, 2023.
(60) Provisional application No. 63/499,894, filed on May 3, 2023.
(60) Provisional application No. 63/503,369, filed on May 19, 2023.

(51) Int. Cl. C12M 1/36; C12M 1/00; C12M 1/32; C12M 3/00

(52) U.S. Cl. CPC C12M 41/48; C12M 23/12; C12M 23/42; C12M 23/46; C12M 29/18; C12M 29/20; C12M 41/06; C12M 41/14

(57) ABSTRACT

A system for conducting antimicrobial susceptibility testing comprising a reader, one or more cassette slots designed to accept a cassette and operatively associated with the computing device, one or more circuit boards operatively associated with each cassette slot, each circuit board including one or more light sources secured to the circuit board, and one or more light sensors secured to the circuit board, one or more cassettes operatively associated with the one or more cassette slots, each cassette including a housing to secure cassette components, one or more well carrier channels within the housing, one or more well carriers which will be housed in the one or more cassettes, each well carrier including a feed line including a lumen, a port secured to one end of the feed line, and a plurality of reaction wells operatively associated with the feed line and open to the lumen of the feed line.

### FIELD OF INVENTION

The present invention relates to a system and method for the rapid detection of the presence and/or concentration of analytes and microorganisms in liquid media using optics.

### BACKGROUND OF THE INVENTION

Antibiotic resistance (AR) and related infections have been increasing over several years and causes more than 2.8 million infections in the US each year. The World Health Organization (WHO) estimates that bacterial AR was directly responsible for 1.27 million global deaths in 2019 and contributed to 4.95 million deaths. Antibiotic-resistant or drug-resistant N. gonorrhoeae is listed as an urgent threat in each of the two Centers for Disease Control and Prevention (CDC) AR Threats Reports (2013, 2019 at cdc.gov website). Urgent threats are the highest of multiple tiers.

Sepsis is the medical term referring to systemic infection that has reached the circulatory system (bloodstream) or other tissue or joints such that septic shock may ensue, eventually resulting in death if untreated. It is generally recognized that the best way to address AR and Sepsis is by new and improved treatments and diagnostic tools. Because it has become more difficult to develop new classes of antibiotic treatments, it is expected that AR infections will continue to rise.

The most widely used methods to conduct applied environmental microbial testing and, or diagnostics include genotypic and phenotypic methods. Specific applications or examples include, but are not limited to nucleic acid amplification tests (NAATs), cell culturing or plating, biochemical testing, or mass-spectrometry.

In addition to accuracy, sensitivity, repeatability and reproducibility, when evaluating applications for use, other factors such as automation, cost per sample, time to result, the need or cost and time associated with sample preparation, and space utilization are often key criteria for determining which application is best suited for a certain investigation or testing or diagnostic.

The term “phenotype” refers to the expression of a gene or genes. Upon expression, the phenotype may be measured or observed in some way. The measurement or observable differences or changes in cells, tissues, or an organism is often used in diagnostics.

Because a phenotype can be influenced by many genes and gene mutations (documented and undocumented), epigenetic and other factors, post-transcript and post-translational modification, as well as a host of environmental factors, phenotype cannot be predicted from genotype with certainty. In the case of antimicrobial susceptibility testing (AST) it is recognized that those tests that utilize phenotypic results are preferred to inform treatment. Thus, with regard to AST and diagnostics, phenotypic testing is understood to be the gold-standard amongst practitioners, such as clinical microbiologists, in addition to being at the core of current automated and high-throughput systems.

AST testing includes many practices, methods, and forms including those that are considered manual methods (typically utilizing liquid media or agar plating) and those that are automated. Classically, with the aid of relatively simple and straightforward microbial cultivation systems or methods, viable bacterial cells are cultured to yield large numbers of clones that can be used for manual or automated analysis. The cultures represent isolates of bacteria that are suspected to be the causative agent of an infection.

The current gold standard AST methodologies for routine medical microbiology laboratories are culture based agar dilution (AD) and broth microdilution (BMD) testing methodologies. Both AD and BMD can determine AST and minimum inhibitory concentration (MIC) and are widely used globally.

AD utilizes molten agar mixed with various antibiotics at different concentrations plated or poured into petri plates. After cooling and solidifying to create a solid substrate, cultured isolates are added and observed for growth or absence of growth after an incubation period. Interpretation of plates is typically completed manually by trained laboratory staff.

BMD refers to liquid suspension of an isolated culture in the presence of varying types and concentrations of antibiotics. The liquid cultures are incubated for a period of time at which the turbidity or cloudiness of the suspension is measured either visually, optically or using other detection means. The turbidity is caused by bacteria multiplying and/or a “growing” culture (resistant), whereas a susceptible culture or isolate would appear as clear or lacking turbidity. Use of a reading device permits this process to be semi-automated.

Alternative manual methods include Disk Diffusion (DD) that is also widely used on a global scale. Easier to perform than AD and relatively inexpensive, DD is a central tool in many laboratories. For the most part until recently, DD required human interpretation. Nevertheless, there are (semi-) automated systems for DD that are available. These systems provide plate reading and automated translation of inhibition zones to susceptibility categories as defined by the Clinical and Laboratory Standards Institute (CLSI) and the European Committee on Antimicrobial Susceptibility Testing (EUCAST). This technology is used in low- and middle-income countries. DD is largely a manual method and cannot provide an estimate for a MIC.

The use of antimicrobial gradient strips (e.g., ETEST by bioMerieux or MIC Test Strip by Liofilchem) is similar to DD, a direct MIC determination can be determined, which is often a requirement. Nevertheless, these methods are manual and require human interpretation.

Despite the existence of numerous automated systems, there are no automated phenotypic AST systems or methodologies existing for determining AST or MIC of Neisseria gonorrhoeae. Gonorrhea infections are typically caused by the pathogen Neisseria gonorrhoeae (NG). The current standard practice for gonorrhea AST employs AD, which is entirely manual, inefficient, resource intensive, and therefore the analysis of is often limited to reference labs where results require a week turnaround time, and human interpretation of the results. Although AST is desirable for treatment, it is not currently practical as it is prohibitively expensive and requires intensive and expert technician time. Because of this, there is very limited testing on NG infections for use in treatment. Today, in the US, only a few sources exist for gonorrhea AST testing for treatment purposes thereby limiting it to large institutional or government facilities such as State-level Public Health Laboratories, select reference labs, Centers for Disease Control and Prevention and Mayo Clinic.

The most widely practiced NG AST testing is conducted for surveillance purposes by the United States Government's Gonococcal Isolate Surveillance Project (GISP) and Enhanced GISP (eGISP) programs that are managed by the CDC. The programs are only able to conduct testing on roughly 2 percent of reported gonorrhea cases annually and the surveillance report is summarized and published annually, which is used as a basis for treatment guideline changes and recommendations.

### SUMMARY

There are several sample types that contain analytes that can be detected. Analytes can include chemicals, biologicals such as microorganisms, proteins, enzymes, or other analytes of interest that can bring a change in the optical properties of the sample being tested. The testing and analysis may be needed to determine the presence of a specific analyte, changes to the analyte based morphology, size, or certain reactions (chemical or biological), and determine whether a biological analyte such as microorganisms are susceptible to chemicals such as antibiotics. Microorganisms can refer to various bacteria, yeasts, molds and viruses. These can be either pathogenic or nonpathogenic in nature.

Herein we describe a system and method that utilizes optics, hardware, software, and fluidic cassettes with single or plurality of wells. We describe a system and method that enables the rapid testing of samples for determination of concentration of antimicrobials inhibiting microbial growth, or the presence or absence of an analyte of interest, or determine changes to the sample over time and/or the relative concentrations or absolute quantities of analytes in a solution or suspension, with options to mix the sample periodically or continuously and or to modulate temperature, humidity, and or gas environment(s).

In one embodiment of the instant invention, the system provides necessary conditions and is customizable to culture microorganisms including, but not limited to fastidious and non-fastidious bacteria, yeasts and molds in a unique environment that allows for their growth, death, stasis or relative changes of different kinds as responses to their environment or other microorganisms (e.g., bacteriophage or fungus) within the fluidic cassette's wells. The system provides the controls for the manipulation of the environment, while the changes to organisms are tracked using optics and software that make up the system. The system can be used to analyze light scatter from each discrete well: over a period of time; variable in the liquid; or relative to other wells. The system can also be used to measure light scatter from wells sequentially over time. Analytical endpoints include using light scatter to measure growth/no growth of microorganisms over time, change in metabolites or reporter-genes, other nutrient changes or colorimetric changes within the sample, changes to scatter intensity, scatter angles, or combinations thereof that can be used to reach meaningful conclusions. Absolute values, relative values, or values changing over time can be used to deduce conclusions.

The system provides the following features: (1) data acquisition of multiple types, both simultaneously or in sequence across one or more wells; (2) software, firmware, or hardware control of data acquisition, periodicity, and system parameters; (3) Data acquisition periodicity can be modulated via software controls; (4) Environmental conditions including temperature and atmospheric gas (e.g. CO₂, O₂, N₂) modulated in a chamber with controls. (5) Mixing the sample at any desired time during processing and analysis (6) Hot-swappable reader units that ‘read’ or acquire data from each cassette.

Thus, the system can be used for a wide range of applications in detection of microorganisms from bodily fluids, food samples, swabs, liquid or other solid samples suspected of contamination or infection from microbes. One such application is in the antimicrobial susceptibility testing (AST) analysis on microbes and pathogens including but not limited to yeasts, molds, mycobacteria, bacteria, fastidious and nonfastidious organisms. Notably this system is the world's first AST system that enables phenotypic testing of Neisseria gonorrhoeae (NG), a fastidious organism and the pathogenic causative agent of Gonorrhea or Gonococcal infections.

The system's wide range of applications relies upon the ingenuity of the method. As alluded to, the present method involves the tracking of changes to light by optics. In this instance, light is defined broadly, to mean any energy form capable of being emitted and detected as waves. Single or numerous wavelengths of incident light enable detection of scattered light across single or multiple wavelengths, with the ability to measure different endpoints for multiple applications, including (a) culturing microbes in a typical or unique environment and detecting and measuring of presence or absence of microorganisms continuously or discretely over time using our instrument, consumables, and software (b) to conduct phenotypic (versus genotypic, molecular, or NAATs) AST on NG and other microorganisms generating Susceptible/Intermediate/Resistant (S/I/R) interpretations and/or minimal inhibitory concentration (MIC) of antibiotics, (c) for identification of the microorganism in the sample based on culturing in the presence of combination of selective medias across different wells within the cassette and (d) other applications where the fluidic sample property changes over time or in a unique environment detected optically.

The invention is a system that includes hardware; a consumable unit referred to as a “cassette”; firmware to control the hardware; software that includes graphical user interface (GUI), data processing, real-time data presentation, database and reporting function(s). The present invention is designed to be compact, scalable, and very low cost and can be built using 3-D printed parts, simple PCBs, electronics and Pi processors. The consumable cassettes are also designed to be easy to 3D print or mold, making it inexpensive as a single-use, disposable consumable.

### DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1A illustrates one embodiment of a reader of the instant invention.

FIG. 1B illustrates one embodiment of the scatter path/detection of the instant invention.

FIG. 2 illustrates one embodiment of a reader of the instant invention.

FIG. 3 illustrates one embodiment of a reader of the instant invention.

FIG. 4 illustrates one embodiment of a reader of the instant invention.

FIG. 5 illustrates a proximal view of one embodiment of a cassette of the instant invention.

FIG. 6 illustrates an exploded proximal view of one embodiment of a cassette of the instant invention.

FIG. 7 illustrates a side view of one embodiment of a cassette of the instant invention.

FIG. 8 illustrates a side view of one embodiment of a well carrier of the instant invention.

FIG. 9 illustrates a proximal view of one embodiment of a cassette of the instant invention.

FIG. 10 illustrates a proximal view of one embodiment of a reader cassette slot of the instant invention.

FIG. 11 illustrates a proximal view of one embodiment of a reader cassette slot of the instant invention.

FIG. 12 illustrates a proximal view of one embodiment of a reader cassette slot of the instant invention.

FIG. 13 illustrates a proximal view of one embodiment of multiple reader cassette slots of the instant invention.

FIG. 14 illustrates one embodiment of the cassette being loaded into a reader, enclosed in one embodiment of the instant invention.

FIG. 15 illustrates one embodiment of the instant invention.

FIG. 16 illustrates a table plotting McFarland Standard Testing: Multiwell vs. Monosystem.

FIG. 17 illustrates a table plotting a McFarland Serial Dilution.

FIG. 18 illustrates a table plotting a McFarland Serial Dilution.

FIG. 19 illustrates a table plotting a McFarland Serial Dilution.

FIG. 20 illustrates a table plotting an NG ATCC 49226 Serial Dilution.

FIG. 21 illustrates a table plotting an NG ATCC 49226 Serial Dilution.

FIG. 22 illustrates a table plotting NG AR 0165 with Ciprofloxacin.

FIG. 23 illustrates a table plotting NG AR 0179 with Tetracycline.

FIG. 24 illustrates a table plotting NG 49226 with Ciprofloxacin.

FIG. 25 illustrates a table plotting E coli 25922 with Ciprofloxacin.

FIG. 26 illustrates a table plotting NG QC Strain (ATCC 49226) in a 5-well cassette system.

FIG. 27 illustrates a table plotting NG AR 0187 with Cefixime.

FIG. 28 illustrates a table plotting S. aureus ATCC #29213 with Tetracycline

FIG. 29 illustrates a table plotting E. faecalis ATCC #29212 with Vancomycin

FIG. 30 illustrates a table plotting K. pneumoniae ATCC #700603 with Imipenem

FIG. 31 illustrates a table with NG ATCC #49226 across 5 wells as growth vs. blank samples

FIG. 32 illustrates a table with NG ATCC #49226 measured in single wells.

FIG. 33 illustrates a table plotting results from a patient positive blood culture where Ampicillin was tested against a clinical positive blood culture sample containing pathogen Serratia marcescens.

FIG. 34 illustrates a table plotting results from a patient positive blood culture where Ampicillin was tested against a clinical positive blood culture sample containing pathogen Staphylococcus aureus.

### DETAILED DESCRIPTION

The present invention now will be described more fully hereinafter in the following detailed description of the invention, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has a range of individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The embodiments set forth below represent the necessary information to enable those skilled in the art to practice the embodiments and illustrate the best mode of practicing the embodiments. Upon reading the following description in light of the accompanying figures, those skilled in the art will understand the concepts of the disclosure and will recognize applications of these concepts not particularly addressed herein. It should be understood that these concepts and applications fall within the scope of the disclosure and the accompanying claims.

The instant invention includes a system and method that utilizes optics, hardware, software, and fluidic cassettes with multiple wells. The system provides necessary conditions and is customizable to culture organisms including, but not limited to fastidious and non-fastidious bacteria, molds and yeasts in a unique environment that allows for growth within the cassette and whose growth can be tracked using the instrument. The system can be used to analyze scatter from a sample in each discrete well. Scatter is generated by directing electromagnetic energy from an emitter into the well where it is reflected and distributed into parts from the whole. The system can also be used to measure scatter from wells sequentially over time. Microorganisms can refer to various bacteria, yeasts, molds and viruses. These can be either pathogenic or nonpathogenic in nature. Light refers to a broad spectrum of electromagnetic wavelengths ranging from UV, through visible, to IR wavelengths. Analyte can refer to any agent, chemical, particulate, protein, enzymes, biologicals such as microorganisms, proteins, enzymes, or other analytes of interest that can bring a change in the optical properties of the sample being tested.

Examples of applied analytical endpoints include using scatter or any shift or change in scatter spectrum from a group, series, or discrete wavelengths to monitor or measure such things as growth/no growth of microbes over time, change in a metabolite(s) or measure expression of a reporter-gene(s), other nutrient changes or colorimetric changes within the sample, changes to scatter intensity, scatter angles or combinations of the aforementioned that can be used to measure changes in a sample.

The system can also support AST analysis on other microbes and pathogens including but not limited to yeasts, mycobacteria, and fastidious organisms. Notably this system is the world's first automated antimicrobial susceptibility testing (AST) system that enables phenotypic testing of Neisseria gonorrhoeae (NG) the pathogenic causative agent of Gonorrhea or Gonococcal infections.

The invention is a system comprising hardware, consumable referred to as a “cassette”, firmware to control the hardware, software that includes graphical user interface (GUI), data processing, real-time data presentation, database, and reporting function. In some embodiments calibration may occur using software or firmware or an external cassette. The instant invention is designed to be very low cost since it can be built using 3-D printed parts, simple PCBs, electronics, and processors. The consumable cassettes are also designed to be easy to 3D print or mold making it inexpensive and a single-use, disposable piece.

The invention includes a method for rapid detection of microorganisms within the system using an optical scatter method. The method can also be employed for detecting changes in the sample not consisting of microorganisms, where a change in the sample properties such as, but not limited to density, or physical or chemical composition over time in the presence of modulated temperature and humidity can be detected by changes in the scatter signal.

In the instant invention, single or multiple wavelengths of light are used conferring the ability to measure single or multiple endpoints for various applications, including:
(a) Culturing microbes in a typical or unique environment and detecting and measuring the presence of microorganisms continuously or discretely over time using the disclosed instrument, consumables, and software;
(b) Conducting phenotypic antimicrobial susceptibility testing (AST) on NG and other organisms including fastidious organisms generating Susceptible/Intermediate/Resistant (S/I/R) interpretations and, or minimal inhibitory concentration (MIC) of antibiotics;
(c) Other applications where the fluidic sample property changes over time, allowing it to be detected optically;
(d) Any application whereby it is desirable to monitor the change or difference of one solution or suspension relative to other solutions or suspensions or over time whereby multiple solutions or suspensions can be analyzed simultaneously or continuously or discretely over time.

In one embodiment, the application of the system disclosed herein is to conduct phenotypic antimicrobial susceptibility testing on Neisseria gonorrhoeae (NG). NG is a fastidious organism and a difficult organism to culture in liquid nutrient broth. The instant invention uses a proprietary media designed for the broth culture of NG. Using specific antibiotics, each at multiple concentrations in each well within the cassette and allowing bacteria to culture in the presence of antibiotic, one can determine the phenotypic AST results. Data generated and illustrated in the figures was generated from samples ranging from single wells or a plurality of wells within one or more cassettes.

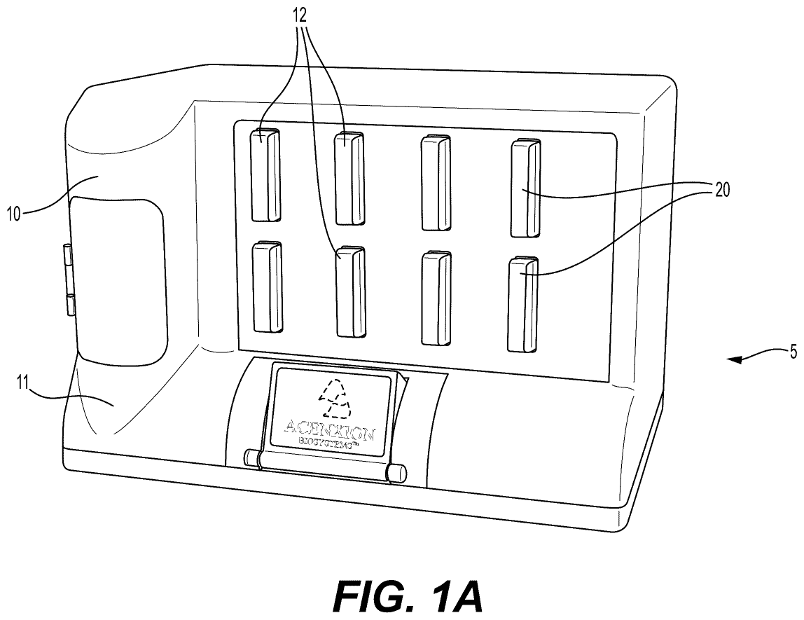

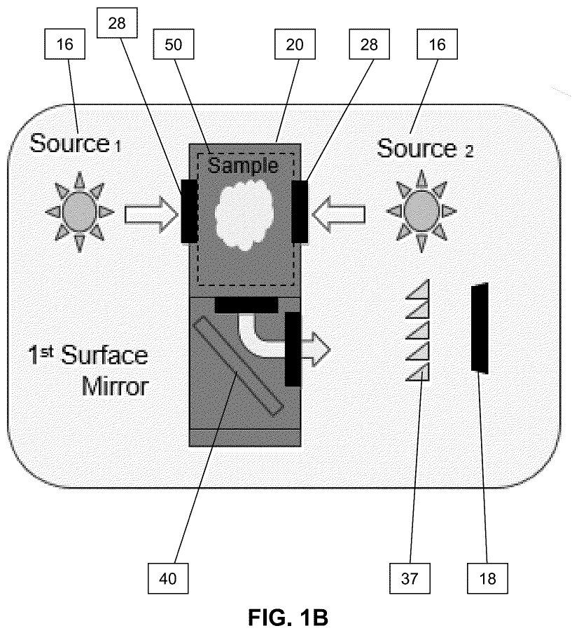

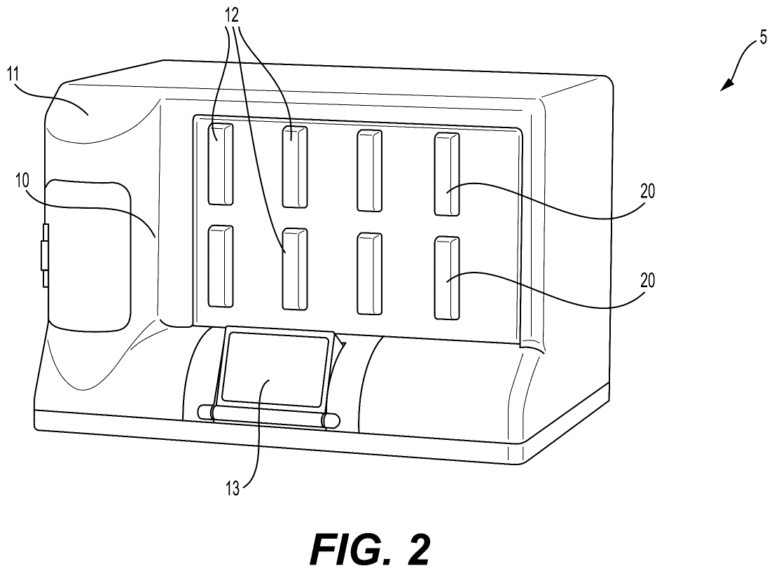

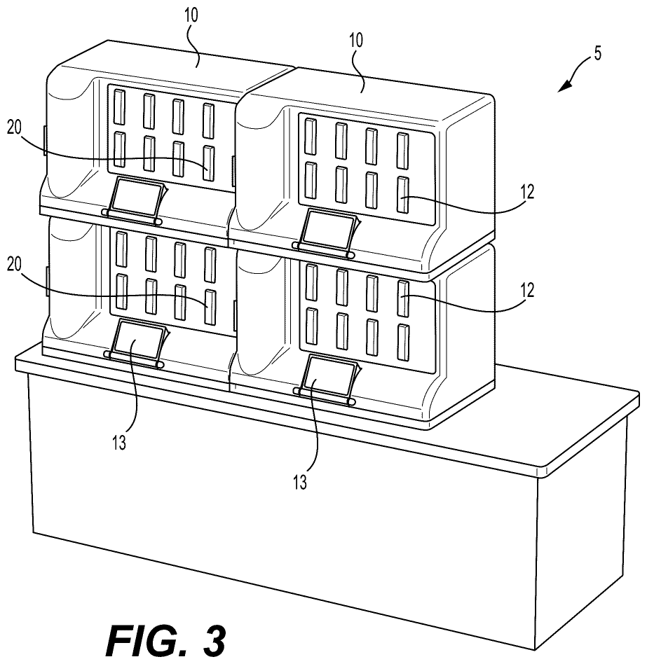

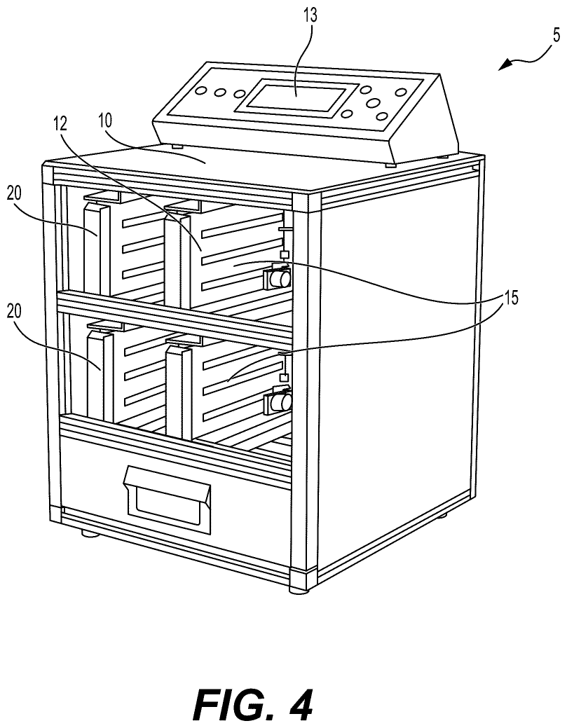

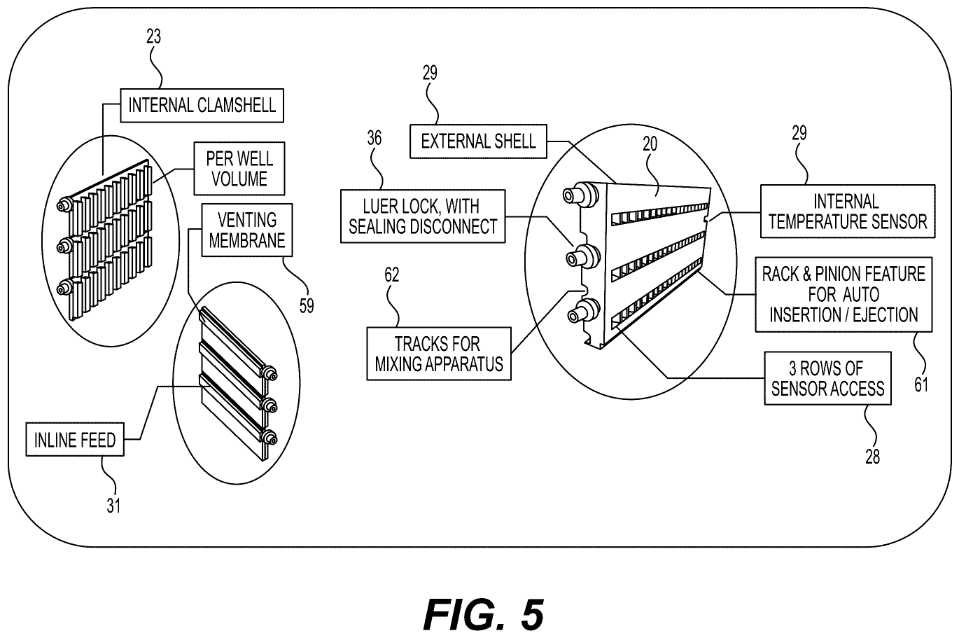

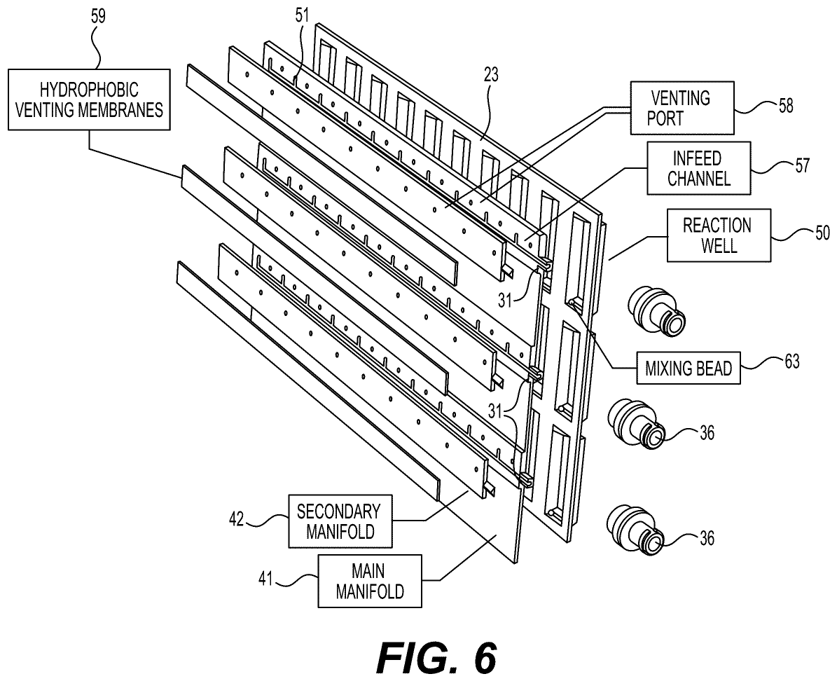

The benefits of the system and method described herein include, but are not limited to:
Automated continuous or discrete measurements can be taken over time in a solution to measure absolute or relative changes attributable to microbial function or change in function
A significant reduction of labor & associated costs by automating the prior art AST process
A significant reduction of labor & associated costs by eliminating the need to create antibiotic agarose plates
The elimination or significant reduction of manual review or interpretation of results & associated costs
A significant reduction of errors associated with transpositions or incorrect or inconsistent interpretation & associated costs
A significant reduction of time to result from roughly one week using prior art technology (Example 1) NG AST system and method described herein AST results for CLSI recommended QC Strain 49226 are typically acquired in less than 3 hours
A significant reduction of time to result from roughly one week using prior art technology to less than 18 hours for difficult strains contained in the CDC/FDA Antibiotic Resistance (AR) Bank that is a collection of difficult isolates obtained by the CDC over the previous decades.
A significant result in consolidating the results from multiple samples to generate custom reports
Significant reduction of waste and costs related to waste disposal
A significant reduction in the space required for analysis
An ability to increase throughput of a high complexity laboratory from accommodating 250 samples per month, to well over 1,000 samples per month.
The system automates labor-intensive process associated with the current art and frees up technical staff time and effort, allowing those personnel to focus on other essential laboratory tasks
The system also significantly decreases the time to report results from surveillance testing for enhanced or improved surveillance, expand or decentralize surveillance testing during automation, and yield annual surveillance reports quickly enabling timely treatment recommendations.
The elimination of the pure culture plating and incubation step to conduct AST on a patient positive blood culture
Significant reduction of time to result for determining targeted antibiotic treatment for patients suffering from bacteremia or sepsis
Flexible design to permit different combinations of emitters and detectors (e.g., Fluorescent sources and detectors, ultra-violet sources and detectors, near-infrared sources and detectors).
The ability to take measurements under varied environmental conditions where heat, humidity, and gas composition may be modulated.
The ability to increase sensitivity is at least partially achieved from the dual light sources, one on each side of the sample, and both are hitting the sample to initially produce 90 degree scatter towards the mirror (reflective surface) and then the mirror directs it to the sensor.
   The sources are equidistant from the sample and located opposite one another from outside the cassette on the circuit board.
One or more filters may be utilized (FIG. 1B) as needed to clarify the scatter measured by the sensor(s).
   One or more optical filters 37 may be used on the light source or light sensor to select for a single or a band of wavelengths.
The system is compact and scalable, via network communication allowing multiple systems to be stacked and controlled by a single host.

The instant invention includes a system 5 that can be used for conducting antimicrobial susceptibility testing, for culturing and/or detecting analytes, comprising a reader 10 which includes one or more processors, a computer readable memory, and a computer readable storage medium operatively associated with a computing device 14. The reader 10 also includes one or more cassette slots 12 designed to accept a cassette 20 and operatively associated with the computing device 14, one or more circuit boards 15 operatively associated with each cassette slot 12 and the computing device 14, each circuit board including one or more light sources 16 secured to the circuit board, one or more light sensors 18 secured to the circuit board, programming instructions to facilitate the growth of one or more samples/specimen within the reader, programming instructions to operate the light sources 16 on the circuit board 15, programming instructions to operate the light sensors 18 on the circuit board 15, programming instructions to record data generated by the light sensors 18, programming instructions to interpret the data generated by light sensors 18, and programming instructions to display the data generated by the light sensors 18 (e.g., on the display 13). The system 5 also includes one or more cassettes 20 operatively associated with the one or more cassette slots 12, each cassette 20 includes a housing 21 to secure cassette components, the housing including an external shell 22 and an internal shell 23, one or more well carrier channels 25 within the housing 21, and a plurality of apertures 28 on the external shell 22 through to the internal shell 23. The system 5 further includes one or more well carriers 30 which will be housed in the one or more cassettes 20, each well carrier 30 including a feed line 31 including an outer surface 35 and a lumen 34, a port 36 secured to one end of the feed line 31, and one or more reaction wells (e.g., a plurality of reaction wells 50) operatively associated with the feed line 31 and open to the lumen 34 of the feed line.

The instant invention includes a system 5 for conducting antimicrobial susceptibility testing, for culturing and/or detecting particulates, comprising a reader 10 which includes one or more processors, a computer readable memory, and a computer readable storage medium operatively associated with a computing device 14. The reader 10 also includes one or more cassette slots 12 designed to accept a cassette 20 and operatively associated with the computing device 14, one or more circuit boards 15 operatively associated with each cassette slot 12 and the computing device 14, each circuit board 15 including one or more light sources 16 secured to the circuit board 15, one or more light sensors 18 secured to the circuit board, programming instructions to facilitate the growth of one or more samples/specimen within the reader 10, programming instructions to operate the light sources 16 on the circuit board 15, programming instructions to operate the light sensors 18 on the circuit board 15, programming instructions to record data generated by the light sensors 18, programming instructions to interpret the data generated by light sensors 18, programming instructions to display the data generated by the light sensors 18 (e.g., on the display 13). The system 5 also includes one or more cassettes 20 operatively associated with the one or more cassette slots 12, each cassette 20 including a housing 21 to secure cassette components, the housing including an external shell 22 and an internal shell 21, a plurality of reaction wells 50 located on the internal shell 21, a primary manifold 41 operationally associated with the internal shell 21, the primary manifold 41 further including one or more feed lines 31 including an outer surface 35 and a lumen 34, a port 36 secured to one end of each feed line 31, a plurality of reaction wells 50 operatively associated with the feed line 31 and open to the lumen 34 of the feed line, a plurality of infeed channels 57 running from the lumen 34 of the one or more feed lines 31 to the plurality of reaction wells 50, with one infeed channel per reaction well, and a plurality of venting ports 38 running from the lumen of the one or more feed lines to the plurality of reaction wells, with one venting port 38 per reaction well. The system 5 also includes a secondary manifold 42 operationally associated with the primary manifold 41, to assist in enclosing the feed lines, one or more venting membranes 39 operationally associated with the secondary manifold 42, and a plurality of apertures 28 on the external shell through to the internal shell.

In one embodiment, the application of the system is to conduct phenotypic antimicrobial susceptibility testing on NG. In one embodiment, our method deploys a series of tightly controlled environmental factors, such as temperature, humidity, CO₂, along with the use of a proprietary defined media while agitating/mixing the sample allowing for the testing of NG. NG is a fastidious organism and a difficult organism to culture in liquid nutrient broth. Using specific antibiotics, at various ranges, antibiotic classes, concentrations across the wells of the cassette and allowing bacteria to culture in the presence of antibiotic, one can determine the phenotypic AST results.

In one embodiment, the application of the system is to conduct phenotypic antimicrobial susceptibility testing on gram positive and gram negative organisms from isolates or directly from a positive blood sample. In this embodiment, our method deploys a series of tightly controlled environmental factors, such as temperature, humidity, CO₂, along with the use of Mueller-Hinton-II defined media allowing for the testing of these organisms as specified by CLSI. Using specific antibiotics, at various ranges, antibiotic classes, concentrations across the wells of the cassette and allowing bacteria to culture in the presence of antibiotic, one can determine the phenotypic AST results.

In one embodiment, the application of the system is to conduct phenotypic antimicrobial susceptibility testing on gram positive and gram-negative organisms from a positive blood sample without plating for an isolate. In this embodiment, the positive blood sample is adjusted to the desired microbial concentration. In this embodiment our method deploys a series of tightly controlled environmental factors, such as temperature, humidity, CO2, along with the use of Mueller-Hinton-II defined media allowing for the testing of these organisms as specified by CLSI. Using specific antibiotics, at various ranges, antibiotic classes, concentrations across the wells of the cassette and allowing bacteria to culture in the presence of antibiotic, one can determine the phenotypic AST results.

The figures show several embodiments of a reader 10 in various configurations. As illustrated, the reader 10 includes a housing 11 and can include one or more cassette slots 12 and a display 13. Multiple readers can be operated independently or as a single unit. The reader 10 can include features such as environmental controls which allow a user to individually control the humidity, temperature, atmospheric gas concentrations (i.e., CO₂, O₂, N₂, etc.), and atmospheric pressure. The reader can also be insulated for temperature, sound, vibration, or a combination thereof. The reader 10 can include one or more temperature sensors operationally associated with each cassette slot.

Additionally, reader 10 utilizes interchangeable source and detector sub-configurations that can be swapped to expand the types and/or range of data collected and analyzed. It is important to note that emitter and detector types do not require consumable redesign. For specific examples, there are configurations that include ultraviolet (UV), visible, near-infrared (NIR) or infrared (IR) emitters and detectors capable of detecting scatter aspects associated with those wavelengths. Another configuration includes the swapping of one or two illumination sources with a second detector component, and in this configuration the same consumable allows for both absorbance measurements and scatter measurements of the samples over time. For samples/specimens that include divergent aspects between their absorbent versus scatter characteristic profiles, additional data and analysis can be performed which capitalize on such divergence. Thus, the system provides an automated means to acquire more information about the sample than would otherwise be known by visible wavelengths alone. While the initial objective and fully implemented device focused on a specific culturing described in more detail below where wavelengths within the visible spectrum provided the data and analysis required for it, these wider scope capabilities are fully anticipated and understood.

Cassette slots 12 are a key component of each reader 10 and each reader can have anywhere from a single cassette slot to a plurality of cassette slots. As stated previously, each cassette slot 12 is designed to accept a cassette 20 and is operatively associated with a computing device 14. One or more circuit boards 15 are operatively associated with each cassette slot 12. Additional components of each cassette slot 12 include a base plate 66 which allows elements of each cassette slot to be secured to it. One or more linear rails 68 may be secured to the base plate 66 to aid in supporting the base plate 66. The instant invention utilizes either an automated or manual insertion and ejection system which is operationally associated with each cassette slot. The automated insertion and ejection system includes components such as an auto insert and eject motor 70, as well as rack and pinion components on each cassette 20. Each cassette slot 12 can include one or more temperature sensors for monitoring the temperature within the cassette slot 12. Each cassette slot 12 can be individually climate controlled. Each cassette slot 12 can further include one or more heatsinks 17 to aid in maintaining the climate controls within each cassette slot and/or within each reader 10 or section of a reader. A rear bulkhead 67 may be secured to the base plate 66 to aid in supporting and enclosing each cassette slot 12.

Each cassette slot 12 can include an insertion module operationally associated with the computing device which includes programming instructions to allow the cassette 20 to be inserted by an operator to a specific point in its movement where the insertion module engages the incoming cassette 20 and takes over the final placement, followed by the precision parking of the cassette 20. Each cassette slot 12 can include a rack & pinion type apparatus operationally associated with the insertion module, whereby a rack gear portion is molded into the external shell 22 of a cassette 20, and the pinion gear is attached to a motor 70 (which is a stepper type motor in the preferred embodiment of the device), whereby the gear and motor portion is physically fixed to the reader frame (e.g., base plate 66). The combination of the motor 70 turning the gear, meshing with the teeth on the incoming cassette 20, pulls the cassette 20 into the reader 10. A sensor arrangement within each cassette slot 12 is operationally associated with the computing device 14 and the insertion module in which the sensor arrangement is used to detect insertion of and movement of the cassette 20. The cassette's movement is detected upon one or more lines located on the external shell 22 of the cassette passing in front of a line detecting component. The sensor arrangement can be a dual channel, quadrature detection, which detects both the leading front edge of lines as well as the rear edge of lines as they pass the detecting component, and by extension of such information can calculate the width. A circuit, processor, and code combination recognize the line's precise location and width. The line and other components combined are used by the insertion module to understand the cassette is being inserted by an operator, where the cassette is in its travel, as well as the speed of insertion. Each cassette slot 12 can include a code which utilizes a line sequence printed on the cassette 20 to park the cassette in a very precise location. The location of the final stopping point such that the optical path of each 90 degree block's light source aperture 28, on the side of the cassette, being near perfectly aligned with its associated light sensor aperture 28. The line sequence can be used for optical alignment to the light sensor aperture during the manufacturing process.

Each cassette slot 12 can include a parking module operationally associated with the computing device and the insertion module which includes programming instructions in which one or more lines are used to first detect the cassette's insertion, then continues at a insertion speed to pull in the cassette 20, waits for a secondary line detection point, whereby the parking module then passes the eventual parking line (a specific line intended to be detected from one edge), such that it is at a very specific location. The initial passing of the parking line occurs by some small minimum distance, where the motor ramps down and turns off and then runs in the opposite direction at a creep speed (very low parking speed), until the leading edge of the line is found in the opposite direction. Upon finding the leading edge the motor is turned off. The cassette 20 is now parked. The underlying accuracy comes from the hardware detector detecting a differential edge configuration associated with the parking module. In one embodiment, once a park line is detected, the parking module could include a primary low speed for X counts, followed by reduced speed. The insert and eject motor 70 (a stepper motor in the preferred embodiment), includes a gearhead arrangement, such that the gear ratio is balanced between having the proper torque and speed to pull in the cassette 20 and having enough stall torque once off to hold the cassette in place during the testing, without allowing movement. The balance of torque vs gearhead includes multiple tradeoffs and one being the ability to have a sufficient pull on the cassette 20 by the operator (more than would be used in any normal operation of the device and only when mandated due to device issues), to overcome the gearheads resistance and remove the cassette manually by force.

Emitters and, or printed circuit boards (PCBs) generate heat and could impact sample temperature. In order to mitigate heat generation, in at least one embodiment includes a means of heat removal. Thermal relief pads were utilized under the emitter sources, and inter and intra PCB mass thermal pathways were incorporated. Multiple intra pathways connected to additional thermal relief pads on the opposite side of the PCBs are used to carry heat away from samples. Elongated heatsinks are horizontally utilized to further transfer the heat from the pads to the system away from the samples. This multi-path thermal connectivity reduces the heat at the sample diffusing and dispersing the heat to the system circulating air.

Each cassette slot 12 can be thought of as a plug-and-play device. Each one can be set up in an individual, specific configuration depending on the microbes or test that need to be completed, and all can run simultaneously, despite their different configurations.

To simplify the design, ensuring a low cost and reduced space requirements, the cassette was designed for easy manufacturability. Therefore it was important to remove any moving components or expensive portions of the analysis from the cassette design. Hence the cassette was designed to be easily inserted and removed and lacks any further connections, incorporating the sample well and optical path.

Several embodiments of cassettes 20 are operatively associated with cassette slots 12. Generally, each cassette 20 includes a housing 21 to secure all of the various cassette components. The housing 21 includes an external shell 22 which encases the various cassette components, and an internal surface. One embodiment of the cassette is a dual clam-shell design. One embodiment of the cassette 20 also includes an internal shell 23 which is housed within and removable from the external shell 23. Cassettes include all elements required to perform a spectral optical measurement within the reader 10. The cassette 20 includes multiple sample measuring support means and structure to eliminate the need for any electronic or electromechanical hardware within the cassette itself, related to the measurement process, other than an optional electronic cassette identification component (preferred version being RFID). Except for such an option and its chemistry within, the cassettes components within are completely passive in nature (non-electronic), making the consumable relatively simple, easily customizable to allow reconfiguration to permit testing for microorganisms, and inexpensive to manufacture by standard molding, thermoforming, RF sealing, ultrasonic welding, thermal bonding, precision adhesive distribution, automated silk screening, or other common technologies used on, consumer based, high volume, products.

A cassette 20 can also include an optical measurement apparatus, whereby the primary functionality is an independent spectral scatter spectrometry measurement performed per sample, via inexpensive, independent, circuitry. A cassette 20 can also include multiple source pathways from different angular directions to illuminate on at least two sides of each sample independently, resulting in an increase in the measurement's sensitivity due to the addition of more direct energy vectors from more than a single side, with those added vectors of energy hitting each particle, resulting in additional dual, perpendicular scatter of what would otherwise have a dark side of a particle with only primary scatter from a single side.

A cassette 20 can also include an optical block apparatus, which increases the reader's 10 sensitivity by reducing undesired side scatter of the reaction well 50 by means of a single part, molded in aperture feature in near-immediate contact with the reaction well 50. The presence of the optical block and its use allow for the cassette 20 to not have any light sensor 18 within or be passed in via an opening. The reason this is so effective is that the alignment between the reaction well 50, the aperture, the pathway, and finally the light sensor 18 are accurately aligned with the only required movements mandated by the cassette 20 to be in one axis and all other components of the reader 10 are fixed in location (i.e., the reader 10 is a fixed block), with the only movement being the cassette 20. The cassette is provisionally parked. The cassette contains optical block reflective surfaces to align sample scatter and detectors. The optical block apparatus increases the reader's 10 sensitivity by reducing undesired reflections from the reaction well 50 itself by its very close (near touching) location to the reaction well 50, reducing reaction well wall 52 reflection into the optical pathway, thus undesirable excess reflections of those walls never reaching the light sensor 18. The optical block apparatus also increases the reader's 10 sensitivity by reducing undesired side scatter of the reaction well 50 by means of a narrowing optical pathway from the mirror to the light sensor 18 such that the reflection of the pathway itself does not add to the signal strength (thus reducing dark current point and increasing sensitivity), due to the angle of the path. The optical block apparatus can be a 90 degree optical block apparatus with a 45 degree mirror element within, which provides for a shorter optical path, other than straight, without the use of lensing, while retaining proper focal distance from the vessel and its specimen, to the detector. This should not suggest a lens could not improve it even more. One embodiment of the 90 degree optical block apparatus with a 45 degree mirror element is configured to allow the mirror to slide or pressed and snapped into place within the cassette. The 90 degree optical block apparatus with a 45 degree mirror element can be a polished stainless steel, or equivalent material, which provides a very thin, flat, rigid, highly reflective, inexpensive, mirror surface. The cassette 20 may also include a means to secure the 90 degree block into the external shell structure via snap fit, to allow for easy and inexpensive assembly, while allowing the combination to be rigid and remain aligned.

The cassette 20 may also include a means to decrease the natural PCB machine lead desensitization effects associated with non-desired far wall reflected energy. The uniqueness in the configuration is that of a 45 degree wall being on the outside of the far back wall of the reaction well 50, thereby creating perpendicular vectors of all energy going out the back wall, rather than reflecting that energy back into the vessel where it would otherwise and unintentionally reach the light sensor 18, resulting in negative effects on the signal to noise of the system. The wall being referenced is located on the outside of the reaction well's far wall, away from the light sensor. Neither a polished, colored, nor textured surface is sufficient to obtain near zero reflected energy. The energy associated with the back wall is energy that is not associated with the primary scatter measurement, thus impeding the sensitivity of the device. As such, this invention includes a mechanical structure which encompasses a unique physical configuration such that the detection mirror of the next reaction well down and near to the back wall area, is utilized as a 45 degree surface to reflect all non-scattered, undesired energy, to the perpendicular cassette wall where it will not reach the light sensor 18. This removes the possibility of degenerative, reflected energy, which would otherwise be measured by the detector, from getting to the light sensor.

Cassettes 20 can also include source apertures for each of the light sources 16. The apertures are sandwiched between the PCB one side with the light sources integrated into the PCB, which allow for wide band LED sources to physically be inside one end of the aperture, while the other end of the aperture tunnel is against the reaction well's 50 transparent wall. This configuration deflects/obscures any external energy into the pathway (forming a barrier), yet fully encapsulates all the desired source energy within, where such energy is focused directly ahead to the sample reaction well. Cassettes 20 can also include a slot for a temperature probe configuration allowing the internal temperature of the cassette, immediately near one of the vessels, to be monitored. Cassettes 20 can also include a shield type housing around the back side of the temperature probe to close off the back side of the cassette as it enters the cassette. This configuration provides a means to more accurately mimic the inner area of all reaction wells, while allowing for the probe to enter. Cassettes 20 can also include an optical measurement apparatus, whereby the primary functionality is to measure discrete spectrum levels and then to combine them into a single color value which includes combined intensity. In one embodiment, such light can be produced by 2700K LED's with wide bandwidth and sharp cutoffs in the undesired energy bands. In such an embodiment, the bandwidth cutoffs may be calibrated to avoid deleterious impacts, while maintaining a flatness better capable of evaluating color. Such a measurement would merge all the energy into the single resulting color value whereby shifts in any specific band level would appear as part of the combined result. Thus, a color shift and its intensity, (i.e., combined overall level pattern) will emerge, and be monitored over time, and be the indicated measurement triggered on.

The external shell 22 of the cassette 20 can have a dual clamshell design. Such a mechanical structure provides for an inexpensive means to have both internal, transparent features, while simultaneously allowing for an outer physical structured layer to supply rigidity as well as non-transparent features. Cassettes 20 can include a single inner clamshell configuration with multiple wells serving as bio growing reaction wells. The cassette 20 can also include an individualized, per vessel inner clamshell configuration, each serving as an individual bio growing reaction well. Cassettes 20 can include individual, transparent clamshell vessels which offer an advantage within the manufacturing and assembly processes. Advantages include the ability to spot or fill with any kind of growth medium 55 or additive or selection media known in the art (e.g., Mueller-Hinton, Lauria-Bertani (LB) Broth, Dulbecco's Modified Eagle medium (DMEM), RPMI-1640 (RPMI); additives such as fungicide, IsoVitalax, vitamins or sugars, serums (fetal bovine serum (FBS)); selection such as antibiotics). Alternatively, the growth medium 55 or additives or selection media may be provided in lyophilized form or a dry formulation.

Cassettes 20 can include the application of an identifier to the clamshell element (vessel), such as a barcode or QR code, to allow temporary or longer term, independent storage, while additional versions are created in a more serial sequence. Thus, allowing for a single, lower cost, assembly apparatus to fill many different vessels prior to final assembly. Cassettes 20 can include individual, transparent reaction wells 50 which include a means of being, mechanically or otherwise, attached to a means intended to fill them. Such an attachment, including a port and sealing mechanism such as an o-ring or aco-molded area, or generically a seal, allows for a leak free connection to the filling line(s). This design can include a means of being mechanically or otherwise, attached to a means intended to fill the reaction wells, including a combined punch and seal feature. Such are commonplace in the packaging industry, and here applied as an assembly means. Cassettes 20 can also include a secondary port, a venting port 58 for the purposes of venting internal atmosphere/gasses such that any trapped gasses can escape as required from the reaction well 50 to fully fill the well. The design can include a venting means, where the well shape and the vent's location are specifically designed to allow “all” of said gas to escape thus by extension ensuring that the vessel can be accurately and efficiently filled to 100% or very near 100% by the incoming liquid. The venting means/port 58 are designed to prevent evaporation and can also include a gas permeable membrane 59.

Other uses of gas permeable membranes within the walls of the wells are important for the sample and act as a primary biosafety containment preventing loss of sample and or sample to sample contamination. By constructing a portion of the well out of a gas permeable membrane that has properties to block liquid loss, increased surface area permits exceptional gas-exchange that may be desired. The main manifold 41, secondary manifold 42, and the reaction well 50 can each be at least partially made of gas permeable membranes.

Each cassette 20 can include a means to fully be degassed prior to sealing. Such would have a near absolute vacuum left behind after such processing to ensure that once the incoming liquid/sample passes through the feed port 51, the port could be opened where the vacuum within sucks the liquid into the reaction well 50 until it is fully filled and excess still at the port. This can be achieved through a first option—infeed is under vacuum too, or a second option which would include a primary vessel off to the side that is used to evacuate the infeed line air. Cassettes 20 can include a defined sandwich structure which allows for: the external shell 22 to retain multiple 90 degree optical block reading elements, retain the inner shell 23 (incorporating the reaction wells), in such a configuration as to allow for all optical paths across the samples to be illuminated by light sources 16 from dual sides through independent apertures 28 on both sides of the external shell 22, while also incorporating the correct optical pathways to allow the light sensor 18 to measure scatter within the reaction wells 50. Light from the light source 16 refers to a broad spectrum of electromagnetic wavelengths including UV, visible, NIR, IR, microwave wavelengths.

The instant invention includes individual cassette wells, Figure X, that utilizes wells and the ability to load a unique sample in each (without feed line), samples may be directly loaded into a well manually or with the use of an autosampler device or liquid handler. The molded wells may be sealed after loading samples and are pressed into the inside of the cassette's outer shell. The two parts of the outer shell are snap closed to form a cassette containing wells and loaded into a reader slot where the reaction wells are analyzed.

In one embodiment, wells can be supplied in rows (we may need a figure) and preloaded with antibiotics or other analytes and may be snapped on and pressed into the cassette's outer shells to make custom panels of different aspects for various applications.

In one embodiment, cassette well surfaces may be coated or contain deposited materials to support microbial growth or function or distribution. Surfaces may be coated with agarose or other matrix to support bacterial culture. Wells could be prepopulated with molten agarose containing bacteria such as referred to as a “bacterial lawn” where bacteriophage activity could be monitored. Wells could be coated with fibronectin and by using mammalian tissue culture media (e.g., DMEM) culturing mammalian cells and similar applications be conducted.

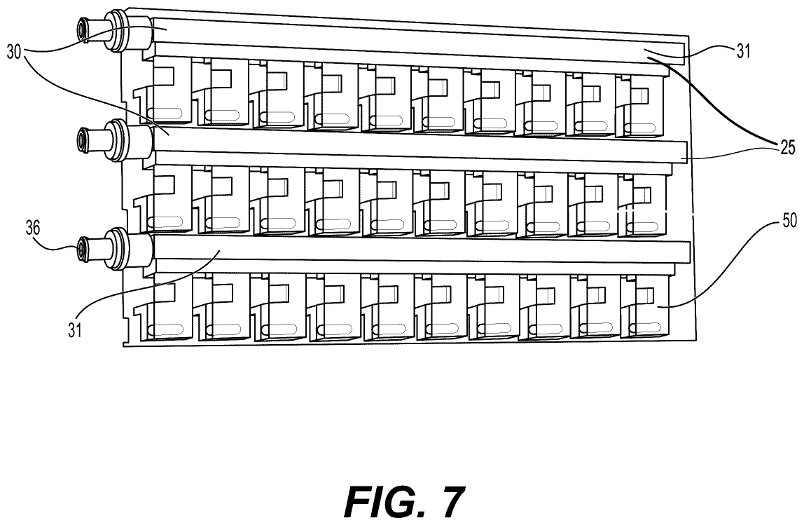

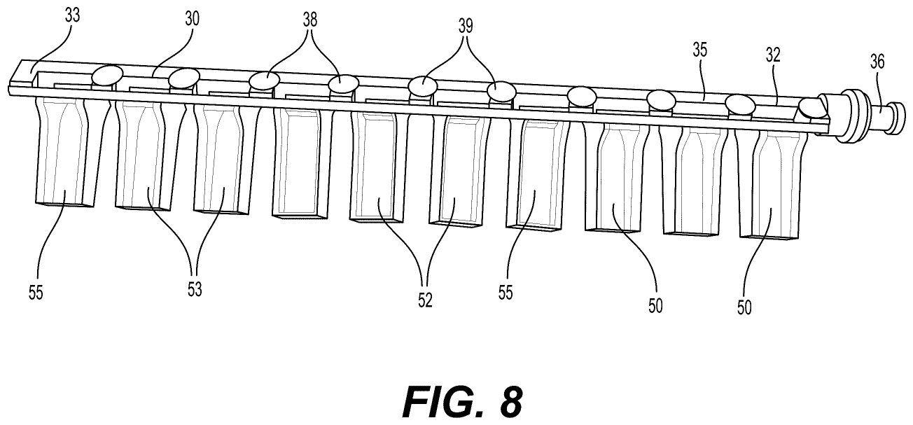

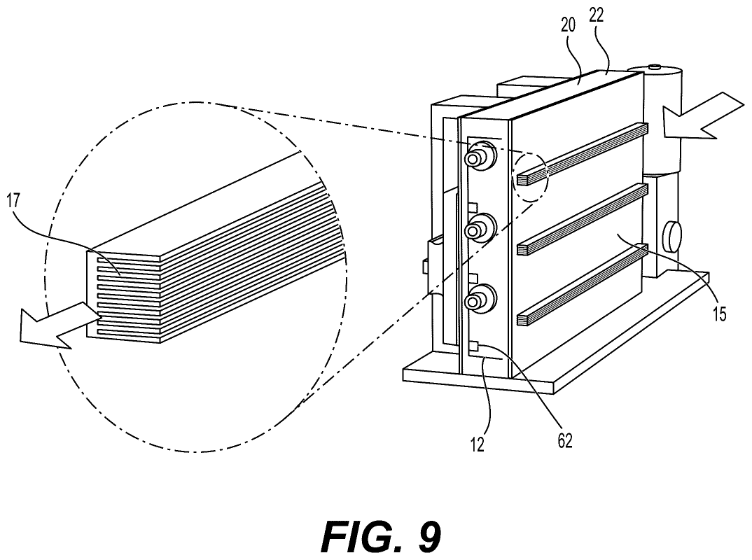

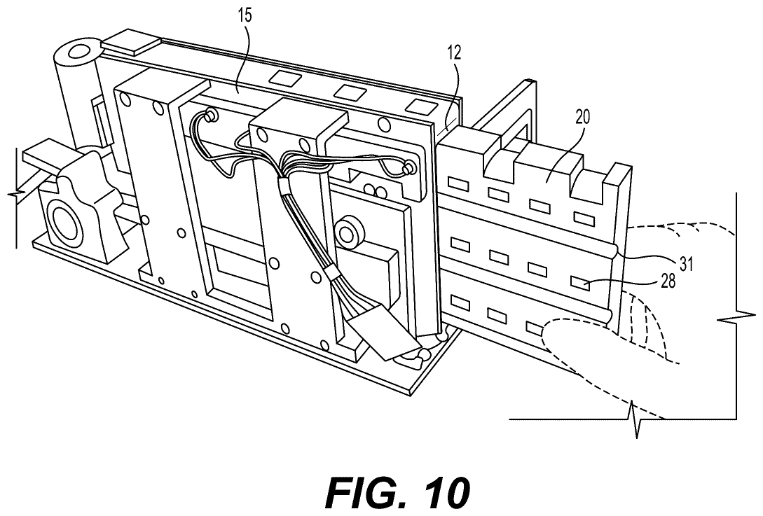

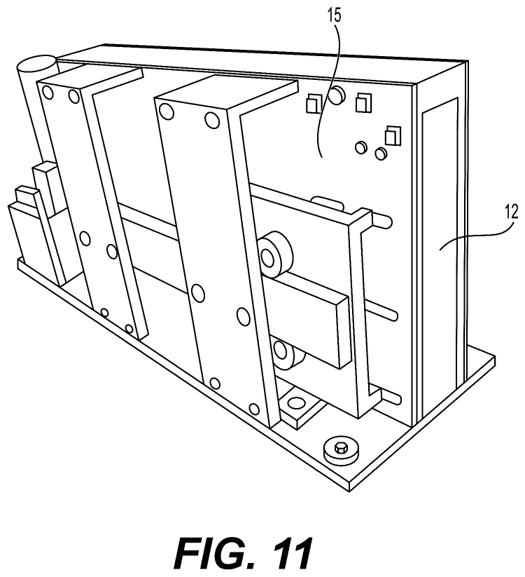

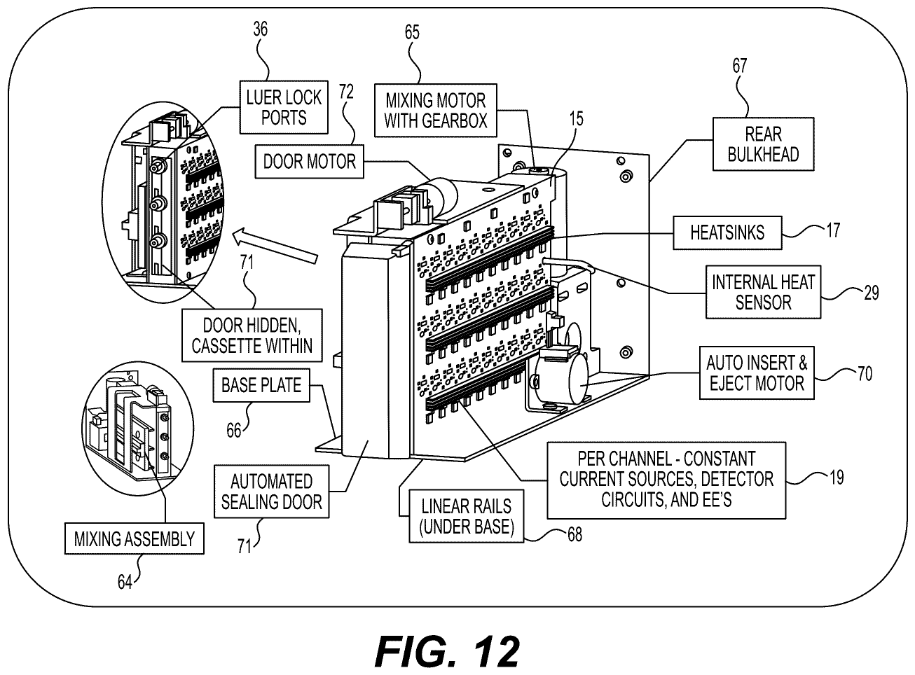

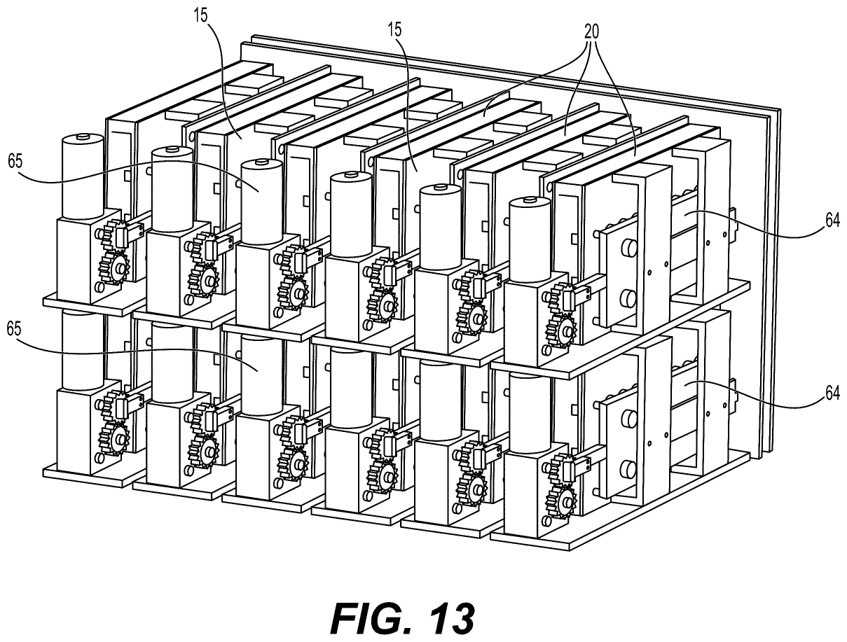

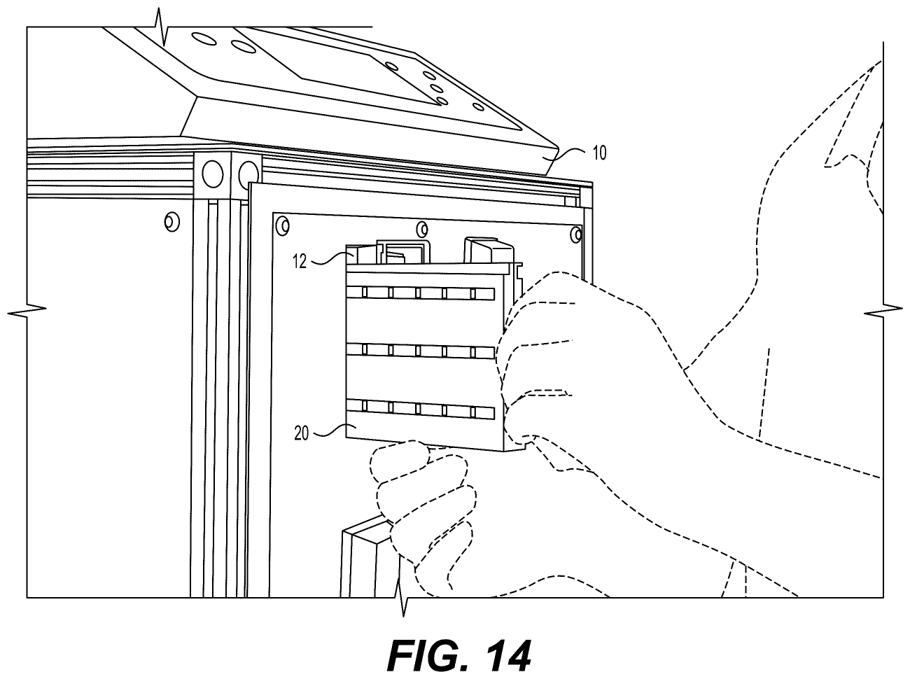

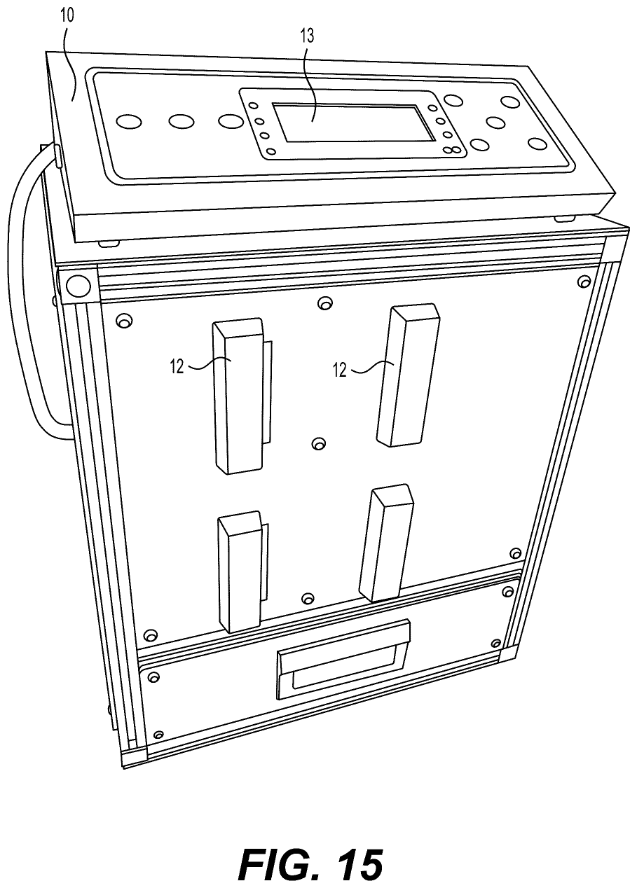

The instant invention also includes a cassette containing one, or more feed lines 31 with one or more pathways each, which provides a means to fill one or more reaction wells 50 from one or more external ports 36. The feed line 31 can be incorporated as part of the internal shell 23, or it can be part of a well carrier 30. FIGS. 7 and 8 illustrate embodiments of a well carrier 30 which includes a feed line 31 including an outer surface 35 and a lumen 34. A port 36 is secured to one end of the feed line 31 and a plurality of reaction wells 50 are operatively associated with the feed line 31 and open to the lumen 34 of the feed line. The port 36 can be a Luer Lock type port. Well carriers 30 can include a check valve seal to avoid contamination escaping during the reaction well filling process. One such check valve example is where the valve is located within the Luer Lock entry point itself, and could be an umbrella version with a minimum cracking pressure such that it snaps shut under a minimum pressure. Well carriers 30 can include one or more breakover valves which inhibit flow until a cracking pressure is achieved within the immediate feed line 31 supplying the reaction well(s). Well carriers 30 can also include a cracking pressure valve per reaction well 50 such that each includes a restricted flow from one to the other after the initial feed line 31 fill pressure is released or bleeds down. Well carriers 30 can include a venting means (e.g., venting membrane) to allow excess air to be evacuated from the reaction wells within such that each reaction well can be filled to the maximum volume within. The material allows gas to escape while retaining the liquid sample within each reaction well. It is important to note that there is more than one venting means (e.g., venting membrane) to handle the evacuation of this air: one is to have a vent per reaction well, a second is to have a single vent at the end of the feed line 31, and a third is to use both to ensure nothing hazardous escapes.

Cassettes 20 can also include a means to mix (mixing assembly 64) multiple sample contents within the reaction wells 50 simultaneously within a cassette. Any mixing method known in the art is acceptable including, but not limited to, vibration, planar motion, mechanical pumping action, Electroosmotic flow, etc. This can be a unified means to substantially mix multiple samples simultaneously within a cassette 20 without electromechanical components in the cassette. This can include an inexpensive unified platform element to structurally retain more than one magnet such that all the magnets can be put in a specific length linear movement stroke, simultaneously, with a single motor element efficiently acting on them as one unified platform. This can include a direct CAM drive configuration to conserve space, provide a specific stroke length, eliminate the need for a crank element, provide direct sensor mounting measurement of stroke speed & home (or park) position. This can include a hall effect sensor acting as the measuring element, which provides a non-contact means, impervious to liquid contamination, and provides a reliable service life.

The mixing bead 63 can be a specific type of bead in the bottom of a narrow channel located within each reaction well 50, which creates the required mixing (e.g., vortexing) action or agitation to mix one or more samples in a linear fashion in a confined space. The round mixing bead 63 being of ferrous type material can be acted on by an external magnet and thus provides the motivative force to provide the mixing action required. Stainless steel alloys are an option as well as oxide treated, or plastic coated versions. Specific strokes per minute provide optimum mixing of the liquid based on its characteristics. The mixing bead 63 can use a specific type of bead, such as, in certain embodiments, a long, neodymium magnet. In certain embodiments the magnetic bead may absorb light, rather than reflect light, to reduce undesired scatter, thus noise within the detectors readings is kept to a minimum thereby producing higher signal to noise ratio. Further, the use of certain materials, as for instance in certain embodiments, may allow for greater coupling with the magnetic bar described while minimizing the coupling between wells. The mixing bead 63 may be in a channel location such that the bead moves linearly below the detection aperture by a minimum distance, and as such any additional scatter produced is at an angle which a combination of the detectors aperture and the optical tunneling of the optical path mask the reflected energy, rather than allowing it to reach the detector. The mixing assembly 64 can include a bead parking means to force the mixing bead 63 in a specific home location to have the least negative affect to the reading by the light sensor 18. When the bead is parked directly under the light sensor's window, scatter produced by the presence of the bead is reduced to its minimum. In such a position the scatter is close to 90 degrees with reference to the sensor's opening as possible. A cavity or indentation below the detector window (parking garage) would help the reduction even more.

The mixing assembly 64 can also include a mixing module including a software and hardware control loop sequence which provides an inexpensive means to regulate the speed and park the magnet holding platform accurately repeatedly, and at the same time lock it in place while reading the samples within the reaction wells 50. The sequence ramps up to a running speed to begin the mixing, runs for the preprogrammed duration of the mixing cycle, then ramps downward to a specific slower speed and waiting for the detection of the hall effect sensor to show it has reached a home position (the hall effect is located at the parking home location), once the hall effect sensor senses it has passed, the system ramps down again to a creep speed (or final parking speed), awaiting for the hall & effect to come around one more time. Due to the creep nature of the last loop, overshoot due to mass and inertia of the motor, gearhead, CAM drive components, platform weight, etc. are negated. Thus only requiring less than one additional turn of the output shaft, at creep speed, to accurately park very repeatedly. Cassettes 20 can also include a series of slots within the consumable cassette to accommodate a very near location between the tip of the narrow magnets and the wall of the vessel holding the sample. Thus, providing for a cohesive coupling distance and very strong interaction between the magnet and bead from the side. Thus ensuring self alignment of bead to magnet in all samples simultaneously across the width of each independent channel upon initial movement in the platform retaining the magnets. Magnets and magnetic beads can be used to isolate, concentrate, or localize magnetic nanoparticles or samples attached to magnetic particles aiding purification or isolation, elution, or sample washing and resolubilization.

The instant system can also include a heat and humidity baffle apparatus. Such baffle apparatus to include means to restrict excess losses of enclosed environmental atmosphere while the testing is underway. The baffle apparatus supplies restrictions while the device has individual cassette 20 access underway in non-synchronized sequence. The baffle may be specifically sized, perforated, and/or membrane dividing elements which provides the restriction between both horizontally stacked as well as vertically stacked readers 10. The baffle may be a material which is a structurally stiff material, and provides the physical mounting component to retain the readers 10 such as steel or aluminum sheet metal. The baffle can be a material which is a structurally rigid material providing the supporting elements for mounting of the readers 10 while simultaneously providing framed areas to retain flexible membranes specifically adapted to the correct inversely related characteristics associated with both temperature control retention as well as providing ample recovery of gas, temperature, and humidity transfer, after the access door is closed and resealed.

The system can include inductive heating of the beads to control the temperature of individual wells within the cassette in addition to or as an alternative to the heat baffle apparatus.

The system 5 can include a delivery device (e.g., a syringe) used during the preparation of samples to be tested by the system which includes in its design and functionality a specialized liquid over pressure protection (OPP) mechanism. One mechanism has a secondary chamber, breakover valve, and vent as part of the plunger section, while another mechanism uses the whole syringe chamber as a recycling area, and plunger divider includes two breakover valves, plus a rear seal. The mechanism allows for all well known syringe functionality, while additively providing protection of the destination device, or an individual, of blowout (i.e. over pressure situation). Such protection being reactive to back pressures associated with the device itself being loaded, and having either a fixed value for such triggering pressure relief, or a dynamic value for such trigger relief. In one configuration, this encompasses two breakover valves. The syringe delivery device can include a secondary containment reservoir or area to capture excess liquid released during the usage of the system. The device includes a means of secondary liquid containment, specifically of the fluid released by the protection means. The syringe delivery device can include a membrane capable of specific hydrophobic characteristics associated with containment of liquids and passing of gasses for the purpose of venting excess pressure within said secondary containment reservoir. The secondary containment reservoir can have an initial volume that is expandable in function, such that it can accumulate the full volume of the primary containment, if mandated to, due to back pressure from the source not being compensated for by initial smaller volume. This can be achieved using a recycled dual breakover design which can be reset with a separate mechanical element added.

The system 5 can include a smart calibration cassette. This embodiment includes an automated calibration means which uses a unique calibration cassette similar to the sample cassette 20, where the calibration cassette internal optical path is programmatically controlled using a smart film or LCD or other chips within the cassette optical path. In this way, the calibration cassette can communicate with the system and auto adjust baselines or thresholds to desired levels or for the purpose of harmonization across one, or selected wells, or a plurality of wells.

Additionally, the system 5 can include one or more dumb-calibration cassettes, to vary the optical path. Other than the different means of controlling the optical path variation, the sequence is the same as the smart calibration cassette. This embodiment includes a calibration means which uses a unique calibration cassette similar to the sample cassette 20, where the calibration cassette internal optical path includes an exogenous material used (e.g., film). In additional embodiments the calibration can be manual. In this way a calibration cassette can be prepared using a standard cassette 20, in which desired solutions or mixtures are used.

For each calibration cassette described above, algorithms can be used to balance all circuits inclusive of light source 16 and light sensor 18. The calibration cassette will mimic the in-process cassette, and includes a block means to mimic the same 90 degree path, a mirror to mimic the small space requirements and allow illumination to enter from dual sides, means to finely select and ultimately to reject portions of the incoming energy to a predefined level. This level will be read by the detection system (light sensor 18) to provide a digital numeric value such that an offset from desired can be calculated. The offset is then applied to either the digital reading on a permanent basis until a future calibration is performed, or adjustment of the initial illumination is performed by offsetting current. A continuous loop is performed to narrow in on a common and desired state per channel. If the loop ends without acquiring a balance, an outer loop will readjust all illumination currents in the opposite direction of the detectors which could not reach the common numeric value originally desired. That is, the system searches for initial settings that will eventually allow for a common energy rejection setting that was initially desired where all channels are balanced. The channel balancing technique means to regulate the amount of illumination per channel such that a specific per band calibration value is obtained per channel.

The data acquisition and analysis means includes a controller and software which provides the logic and sequence which initializes all hardware communication, sequences mixing via a defined speed and time profile, controls light energy levels during the measurement process via independent, programmable, constant current sources, and sequences each measured point of potential growth by taking multiple measurements per reading. Software reading is achieved by multiple readings being taken of the single data points, comparing all readings of that point, and via algorithm eliminating the outliers from the array of data points. The unique algorithm includes weighing techniques for the removal of outliers, temporary evaluation and recording of the delta per point as compared to all other raw values gathered, posting each point set of deltas, using those and all others for all data points, in such a way to find a consensus of deltas. The algorithm retains all raw points which match within a fixed or weighted window, while removing any that fall outside of such consensus. If for some noise or other related reason, there is a consensus less than some minimum number (say 3 for example), the system sets a flag to indicate the data should not be used to detect overall growth. By performing this sequence of events, only consensus data points remain and are utilized, rejecting irregular or noise induced data from the prediction of growth. The algorithm's rejection weighting value can be fixed or weighted based on several techniques, the simplest being a fixed number, which works if the system's overall noise is below a certain experimentally deduced threshold. Multiple measurements are averaged.

The software, in some iterations, also includes an algorithm for “making call” or determining changes in the reaction wells, such as an increase in particle concentration, change in size of particles, or growth of bacteria over time, change in color of the suspension, detection of certain chemicals or biological components, etc, identification of presence of certain chemicals or biological components, etc. The algorithm can use initial data as a baseline, with normalization. By using time zero or initial measurement value (t₀V) as a reference value, the changes in a sample over time can be measured. All bacteria share an understood growth profile (lag phase, log phase, plateau phase, and death phase). Utilizing profile shape analysis in and of itself can provide extensively valuable information, regardless of the absolute or the instantaneous growth point measurements. Analysis associated with the growth profile is an important aspect of an automated system evaluation as it relates to MIC or other comparisons.

In some forms, the algorithm tracks changes to the data from each well over the reaction period/time, in real-time or at frequent intervals. In other forms, the algorithm can use absolute values of the initial data to track changes within each well. In certain applications, the algorithm uses machine learning to track significant changes in the well relative to other wells, while in certain other applications, the change is tracked as an independent variable for each well. In yet other forms, a machine learning algorithm can be given learning datasets based on the sample type to identify trends in noisy data, which can then be used to make predictions on the sample within each well. In certain forms, the algorithm uses a pre-defined equation or combination of equations, to extract meaningful parameters within the equation. These parameter(s), either individually or in combination, can be used to track changes within the well. In additional forms, the equations or the predictions will have statistical analysis performed to make calls on the “significant” changes within the wells and may include confidence levels for such calls. In certain other embodiments, the algorithm may use combinations of preset equations and machine learning to track multiple different parametric changes occurring within each reaction well either with or without relation to other reaction wells. These algorithms may provide an additional level of detail to the changes within reaction wells and/or may add confidence to the result reporting. In some embodiments, an integral part of the system's functionality is the ability to make automated growth/no-growth calls for biological samples. In some embodiments the data collected from the system runs to train and evaluate a support vector classification (SCV) model. SVC is a supervised machine learning (ML) model, as it learns from labeled data and uses it to classify a data set into a category. In some embodiments, a relational database (DB) can be created to collect, organize and store data originating from a network of separate instruments across locations to feed into the ML analysis.

The software for reporting the processed data will be formatted and customized based on the end application for ease of automated reporting, real-time surveillance reporting, communication outside the system etc. A report will be generated automatically by combining the individual results from one or more reaction wells into meaningful groups, interpreting the signal changes and providing a report on the results from each sample within the cassette along with the result interpretation and inferences, if any.

In certain forms such as AST results, the software will combine results from multiple wells which have the same antibiotic but at different concentrations. In some iterations it uses algorithms to make calls on growth or no growth in each well. The software then combines the antibiotic concentrations to the growth patterns for each antibiotic, yielding either S/I/R interpretations (based on documented databases for result interpretation for AST) or MIC values with S/I/R interpretations. The result report can also provide inferences such as “Alert Isolates” based on if the MIC values are high per CLSI or CDC guidelines. In other forms, the results from individual wells will be reported separately and are interpreted as independent results with multiple reports generated per cassette. In yet other forms, the sample may be tracked to identify change in the signal within each reaction well, with one or more reaction well results combined to yield a single result interpretation report. In certain cases, the software can have custom report generation options, to generate reports such as QC, diagnostics, sample testing, etc. from each system or stacked systems based on custom field selection. The custom fields can include choice of cassette type, sample type, result outputs, or any other data or metadata associated with the sample or system. These result reports can be used to infer or track changes to the system over time, changes to the sample processing, quality controls or other diagnostics parameters. They can also be used to summarize result reporting customized using fields over days, months or years.

### Method Workflow:

The workflow involves use of a sample preparation kit (if and as needed) to process the sample to be tested (based on the sample type). There can be additional sample preparation steps with or without the use of the sample preparation kit. The sample is then loaded into the cassette unit using one of several means—injection syringe, pipette or automated loading. The sample identifier, which may be in a barcode sticker consisting of sample details, is placed on the cassette. The cassette has an RFID or barcode ID that encodes the cassette information. Both these are scanned using a RFID and/or barcode scanner on the host computer. The cassette is then loaded into one of the open bays/readers within the instrument. The reader also has an RFID scanner to record the cassette location i.e., the bay/reader it is inserted into within the instrument. The cassette is then autonomously incubated within the instrument, during which all wells within the cassette are read independently by the reader at regular specified time intervals, until the test is complete. The test completion is determined either by the prespecified time period for the cassette to be run, or until the software algorithm running the data analysis in real-time determines the necessary change to the call to complete the test. The host computer or the processor within each reader runs the data analysis from the wells within the cassette in real-time (after each datapoint collection) to determine if there was signal change within each well relative to initial time or a defined time window. Based on the statistical analysis, if the signal change was determined as significant for a given well that well is considered to have detected the analyte. The test can also be used to determine the absence of an analyte if the signal change is insignificant or no change is observed over the testing period. The result report for each cassette is generated at the end of each run summarizing the results and giving result interpretation based on the analyte under test. In the example of the AST application, each well with the cassette, consisting of a plurality of wells, contains different antibiotics at different concentrations. Based on the individual wells growth/no growth determination by the algorithm and defining if the change is statistically different, the result report can be generated to summarize the results to determine the MIC values for each antibiotic, and respective Susceptible/intermediate/Resistant interpretation.

Additional Elements of the System 5 and additional application of the system can include:

### Sample Preparation:

The system can accept a wide range of samples, going from isolates of culture or directly from primary samples with or without sample processing based on the sample type and application. In some embodiments, the sample can be preprocessed to clean it up to remove unwanted, interfering material or contaminants. In some embodiments, the samples can be concentrated to increase the amount of analyte to be detected, while in some other embodiments, the samples may be diluted to either decrease the analyte concentrations or to dilute the interfering materials. The preparation can also include addition of certain chemicals to kill contaminating microorganisms, while keeping intact the target microbe for detection.

### Sample Types:

This system has an input in the form of a (primary) sample, sample mixture, solution, or suspensions originating from swabs, blood draw, urine, other body fluids, environmental, pharmaceutical, food samples, etc. where one aims to detect presence of various analytes that can be optically detected such as but not limited to microbial, mammalian cells, chemicals, nanoparticles or other analyte types. In some forms, the sample is a bacterial colony (isolated from a primary sample and/or solid media). In other forms, the sample is a swab used on a patient to collect microbes from the body. In some forms the sample can be a blood draw or the blood culture vial. In yet other forms, the bacterial sample is grown in a selective media suspension. In other forms, the suspension is created after sample cleanup or pre-processing to remove inert or interfering substances. In yet other forms, the sample can be diluted or enriched using ligands, antibodies, nanoparticles or other means to concentrate the analyte of interest. Thus, the input suspension can be made from a number of different sources or source materials. Furthermore, in other forms, the input suspension is made up of a chemical formulation which at the least does not kill the bacteria and allows for the bacteria to thrive when free of known chemical formulations capable of interfering with the growth behavior of the bacteria suspended. In certain embodiments, the sample can be uncharacterized or semi-characterized nanoparticles where the system can be used to characterize them based on but not limited to nanoparticle type, shape, size, uniformity, quantification or any other properties. In certain other embodiments, the sample solution can be added into the cassette and the process of nanoparticle formation occurs within the individual wells, which can be tracked until completion using the system. With respect to the sample, the input suspension can thus be made with or without sample processing. In certain other embodiments, the sample types can be mammalian cells for application in cancerous or tumorous cells or circulating tumor cells, where specific mammalian cell culture media can be used, and adequate surface area provided for the mammalian cells to grow on within the wells of the cassette.

Sample preparation kit:

The system may consist of a sample preparation kit that is developed unique to the original sample type (for example, Swabs, Urine, Blood, other body fluids, environmental, chemical, mammalian samples, etc.). To create the input suspension, multiple sample kits may be provided requisite to the creation of an input suspension. The input suspension could be prepared with or without the use of a sample kit. In some forms, there will be sample kits which allow the input suspension to be made from swabs, loops or aliquots carrying the sample. In other forms, the kits will allow the input suspension to be made by concentrating or diluting the sample into media. In yet other forms, the input suspension will be made with the use of kits in conjunction with common laboratory equipment as may be available. In further forms, the iterations of the kits will be specifically designed for distinct uses. In some of these forms for which specific designs and distinct uses are made available, the suspension will be made according to the design and distinct use. In some embodiments, there may be customized versions of the kits which accord with the specifications needed to increase the range of samples or sample types to be used by the system. In certain embodiments, the sample preparation kits may have signal enhancing or suppressing components such as dyes, fluorescent chemicals, fluorophores, or uses Forster/Fluorescence Resonance Energy Transfer (FRET) modality, based on the sample type and the analyte to be detected.

### Big Data for Private/Public/Research Use:

Potential use of the local, regional, national and global cumulative data (without any patient identifiers or associations) from the pooled data from systems to report antibiotic trends, resistance patterns, antibiograms, and treatment trends. In some forms of the system, there is data generated either in the use of the system or in the potential for use. This data can be generated with or without actual use of the system and incorporates the data generated by those who may use the system as well as those for whose benefit the system is used. Likewise, in some forms, the system is able to generate data about the data it, itself, produces, called meta-data. This data and meta-data, in some forms, could be pooled or collated. In some forms, the pooled or collated data could contain information related to the sample and the results from using the sample, for example sample type, kind of test completed, or kind of results, including, in some forms, the results themselves, in some forms including interpretations of these results. In yet further forms, it is possible for the system to generate data collections which bring together the pooled or collated data along with the information about the data generated by the system, and bring this data together with data it can access from archives or databases made available through connectivity to networks. These forms could use information provided to the system by the user to networks that contain further data at local, regional, national, or international levels. Thus, in yet further forms, it is possible that the system could generate data that could be accessible to networks of other data and that this access may allow for certain kinds of reports, inclusive of medically relevant reports to be generated and shared to devices and users not directly involved in the use of the system. For example, reports could be generated in some forms of the system that give information about bacteria strains, sample populations, and trends in activity of chemical or pharmaceutical agents. These reports, data or metadata, metadata without any personal identifiers can be shared across research communities, hospital networks, clinic networks, surveillance agencies, governmental agencies, and/or private organizations to help understand trends within or across different sample types, regions, and may guide recommendations of future sample processing and result interpretations, in some embodiments could include treatment guidelines for patients.

Antibiotics in the Cassette where:
a. antibiotics at specific concentrations are pre-loaded in the wells and that
b. antibiotics can be in lyophilized form.

In certain embodiments, the reaction wells may contain liquefied, freeze-dried, powdered, or lyophilized chemicals, reagents, or nanoparticles that come pre-loaded within the cassette. These agents may get rehydrated or diluted instantly or may have a delayed/timed release to react with the added sample suspension. In certain forms, the chemical can be an antibiotic at different concentrations which react with the sample suspension upon addition. This reaction will be tracked over time, which will determine the effectiveness of certain antibiotics or concentrations of antibiotics against the sample suspensions. The concentrations of the antibiotics can be either used at multiple doubling dilutions to determine the MIC value for the pathogen tested, and or chosen to be around the breakpoint values to obtain the S/I/R interpretations alone. In certain other forms, the chemical can be biological enzymes or components that could act as markers for tracking changes to the sample either instantaneously or over a course of time or space. In other embodiments, the pre-loaded material may contain nutrients to aid in enhancement of sample reactions within each reaction well. The chemicals added in certain other embodiments can be used to identify the unknown sample input into the cassette. In certain embodiments, reagents such as nanoparticles, can also be used to characterize, analyze, or make inferences on the analyte samples being input into the cassette. A combination of different chemicals, markers, nutrients, etc. can be present within each reaction well or across different wells within the same cassette based on the analysis needed.

### Direct-from-Positive Blood Culture AST:

Acxbio has successfully demonstrated the potential to address the largest market for AST products: the hospital clinical laboratory and direct-from-positive blood culture AST and conducted in collaboration with University of Kansas Medical Center (KUMC) in Kansas City, KS. The system protocol and results from patients were compared to that of KUMC's FDA-cleared automated Becton Dickinson Phoenix™ System. The system successfully called correct AST results in 3-6 hours, and on average, 35 hours before the KUMC lab clinical instrumentation made the same call. Study samples were provided by KUMC clinical lab and consisted of 23 de-identified and blinded positive blood culture aliquots that were sitting on the shelf to be discarded. KUMC lab conducted Gram staining and identification, and the positive sample was aliquoted for analysis. KUMC continued their standard of care including plating, colony isolation and AST run on the Phoenix. Study included 8 species of bacteria including E. coli & S. aureus, the two most prevalent bacteria in sepsis infections. Summarized results supported that the system eliminated the plating step and arrived at results in <4-6 hrs typically, 35 hours faster than plating (overnight) and BD Phoenix (11-23 hrs) with an overall EA of 97% and CA of 96%.

Decontamination, or Preventative Maintenance is possible using UV lights, or heat or gas.

The instant invention also includes a method for testing a sample comprising the steps of:
(a) providing a system 5 for conducting antimicrobial susceptibility testing comprising:
   a reader 10 which includes:
      one or more processors, a computer readable memory, and a computer readable storage medium operatively associated with a computing device 14;
      one or more cassette slots 12 designed to accept a cassette 20 and operatively associated with said computing device 14;
      one or more circuit boards 15 operatively associated with each cassette slot 12 and said computing device 14, each circuit board 15 including:
         one or more light sources 16 secured to the circuit board;
         one or more light sensors 18 secured to the circuit board;
      programming instructions to facilitate the growth of one or more samples/specimen within the reader 10;
      programming instructions to operate the light sources 16 on the circuit board 15;
      programming instructions to operate the light sensors 18 on the circuit board 15;
      programming instructions to record data generated by the light sensors 18;
      programming instructions to interpret the data generated by light sensors 18;
      programming instructions to display the data generated by the light sensors 18;
      one or more cassettes 20 operatively associated with the one or more cassette slots 12, each cassette including:
         a housing 21 to secure cassette components, the housing including an external shell 22 and an internal shell 23;
         one or more well carrier channels 25 within the housing 21; and
         a plurality of apertures 28 on the external shell through to the internal shell;
      one or more well carriers 30 which will be housed in the one or more cassettes 20, each well carrier including:
         a feed line 31 including an outer surface 35 and a lumen 34;
         a port 36 secured to one end of the feed line 31; and
         one or more reaction wells 50 operatively associated with the feed line 31 and open to the lumen 34 of the feed line;
(b) providing a growth medium 55 within each reaction well 50;
(c) providing a delivery device which is used to introduce samples/specimen into the system, the delivery device containing a sample/specimen;
(d) injecting the sample/specimen into each port 36, which flows into the feed line 31 associated with each port 36, which then flows into each of the plurality of reaction wells 50 associated with each feed line;
(e) inserting the cassette 20 into the cassette slot 12 within the reader 10;
(f) mixing the sample/specimen within each reaction vessel 50;
(g) activating the programming instructions to facilitate the growth of the sample/specimen within the reader;
(h) activating the programming instructions to operate the light sources on the circuit board;
(i) activating the programming instructions to operate the light sensors on the circuit board;
(j) activating the programming instructions to record data generated by the light sensors;
(k) activating the programming instructions to interpret the data generated by light sensors; and
(l) activating the programming instructions to display the data generated by the light sensors.

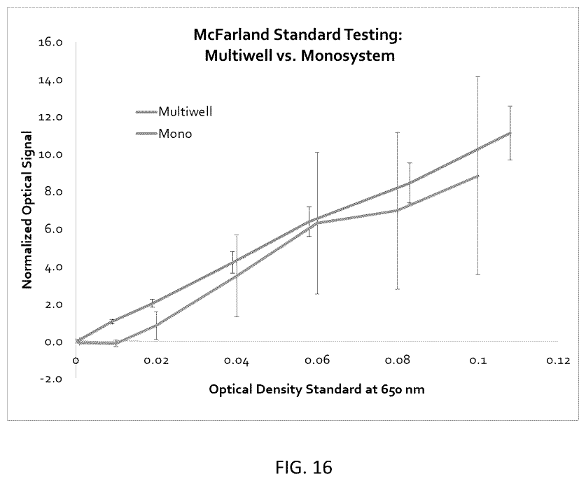

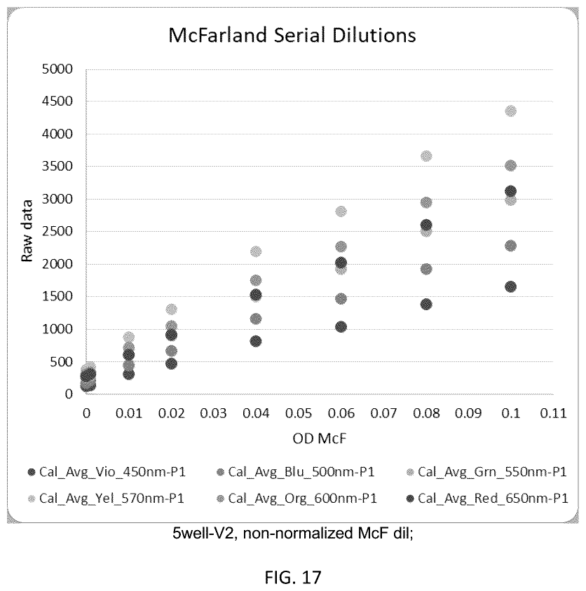

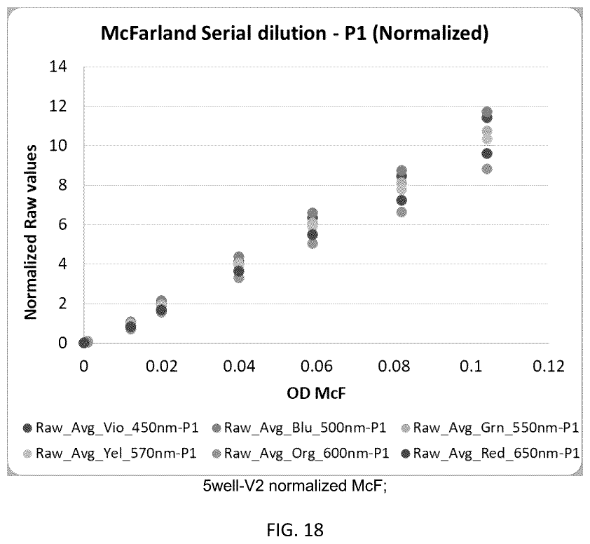

Looking now to FIGS. 16 through 34, the tables illustrate:
FIG. 16: A McFarland stock solution (these are latex bead solutions that mimic optical density of bacteria at different concentrations) was serially diluted to give a optical density range of 0-0.1 (mimicking bacterial concentration of 0-1e8 CFU/mL).
   McFarland solutions are used for initial testing since they are shelf-stable and the engineering team can use this without any biosafety concerns.
   Typically, we expect a linear relationship between OD values of McF and the signal we get from our instrument.
   The standard deviation here is the signal measured across all 6 wavelengths for each point.
FIG. 17: 5well-V2, non-normalized McFarland dilution;
   This Figure illustrates the raw data values from our device (before any data processing) vs the McFarland OD values between 0-0.1 across multiple wavelengths (450 nm to 650 nm).
FIG. 18: 5well-V2 normalized McFarland;
   This Figure illustrates the normalized values from our device (before any data processing) vs the McFarland OD values between 0-0.1 across multiple wavelengths (450 nm to 650 nm).
   Normalized values are normalized to 0 (no McF−DI water only) reading, (i.e., 1st datapoint).

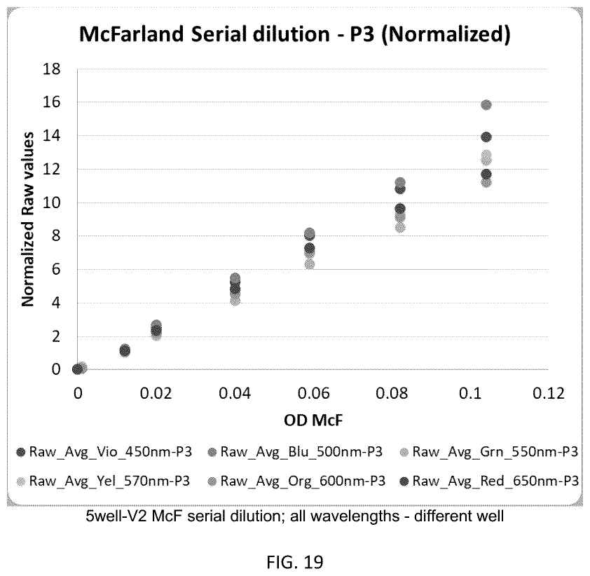

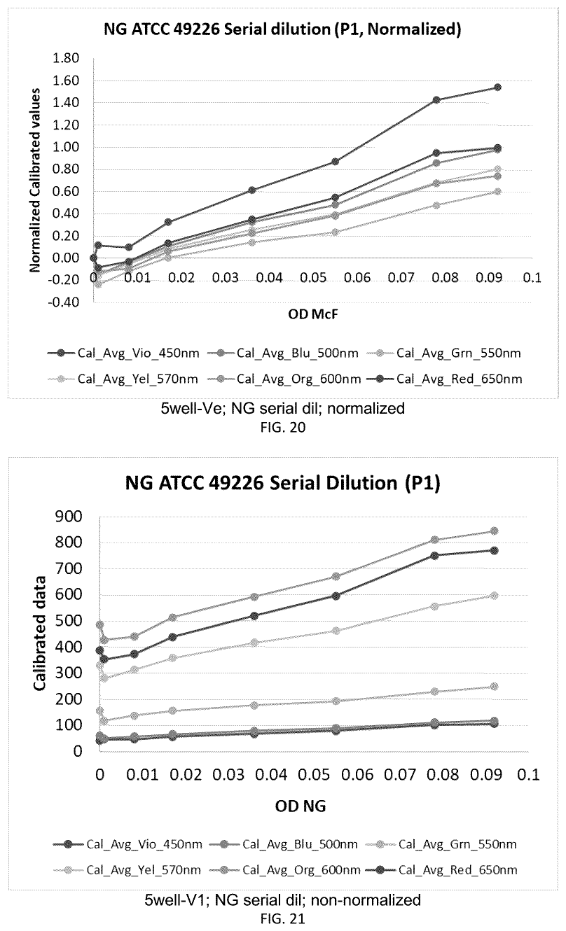

(Readingₓ−Reading₀)/Reading₀ Normalization formula:
FIG. 19: 5well-V2 McF serial dilution; all wavelengths—different well within the same cassette.
FIG. 20: 5well-V1; NG serial dilution—normalized;
   This figure illustrates the normalized values from our device (before any data processing) vs the N. gonorrhoeae in our medium with OD values between 0-0.1 (similar to McF) across multiple wavelengths (450 nm to 650 nm).
   Normalized values are normalized to 0 (no NG—GW media only) reading, (i.e., 1st datapoint).

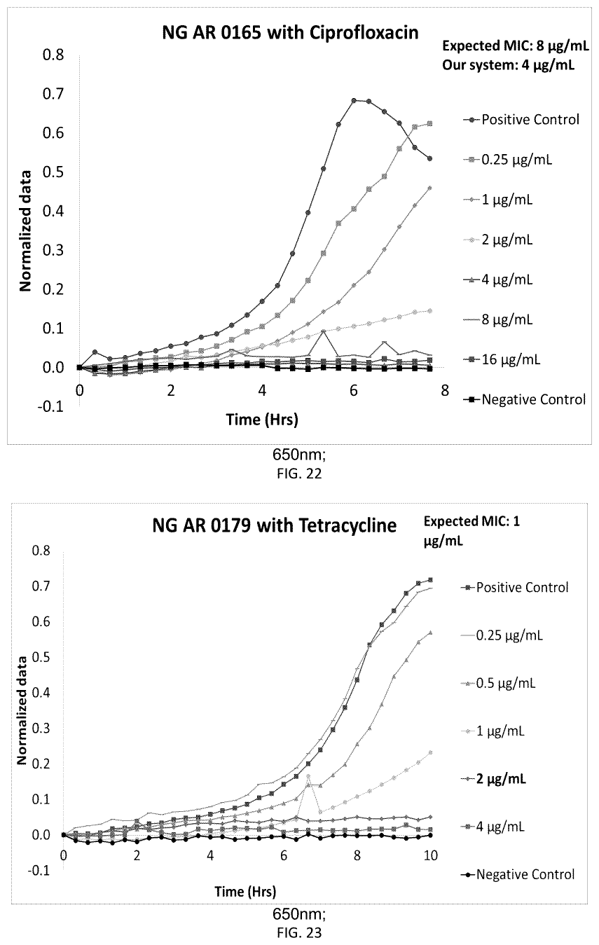

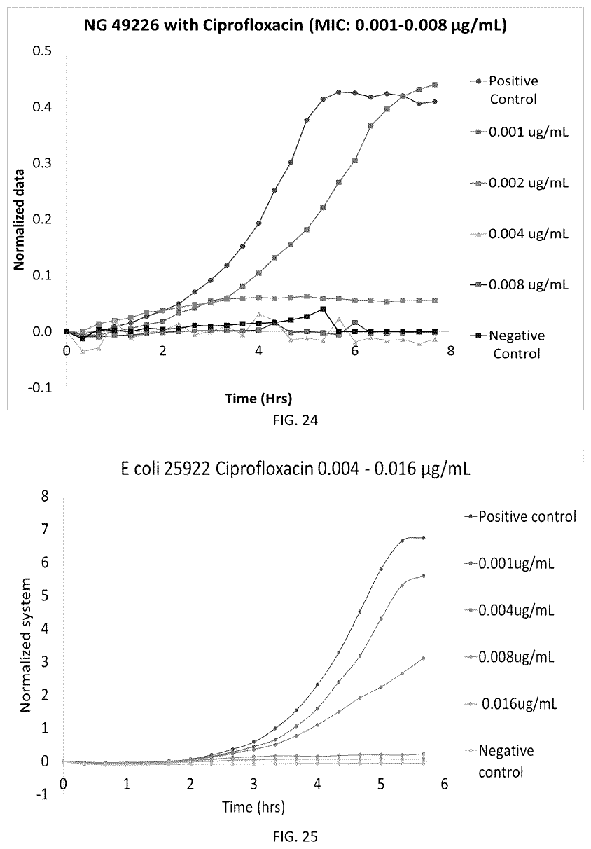

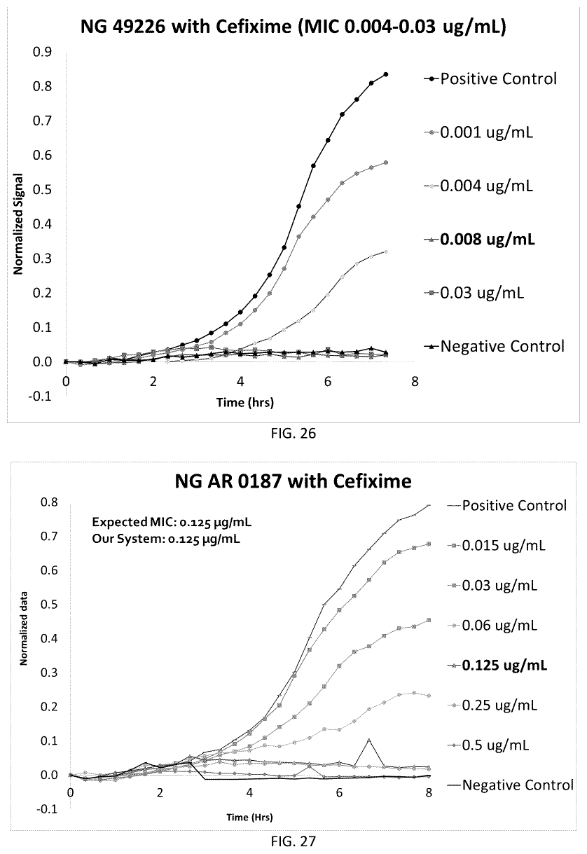

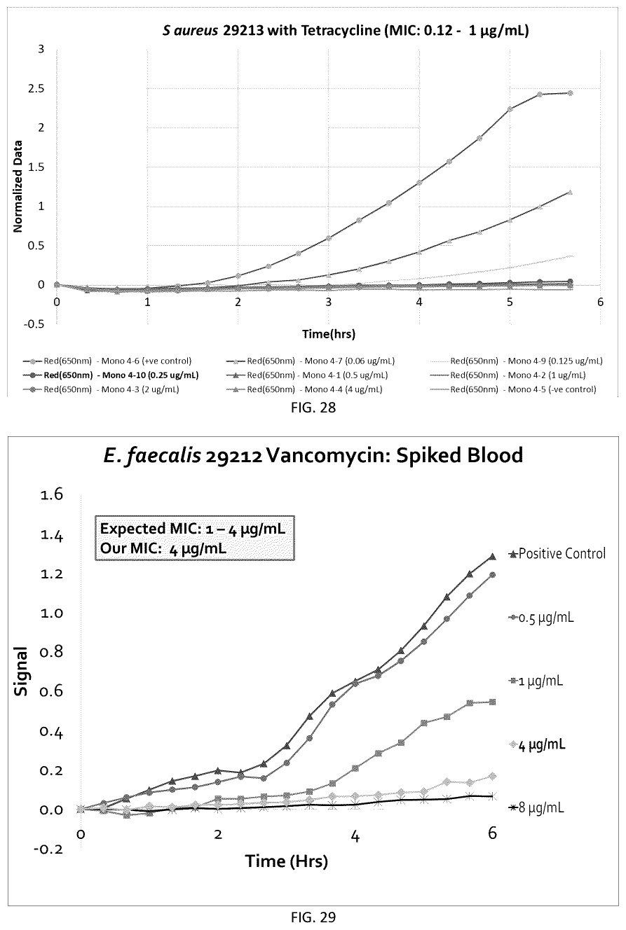

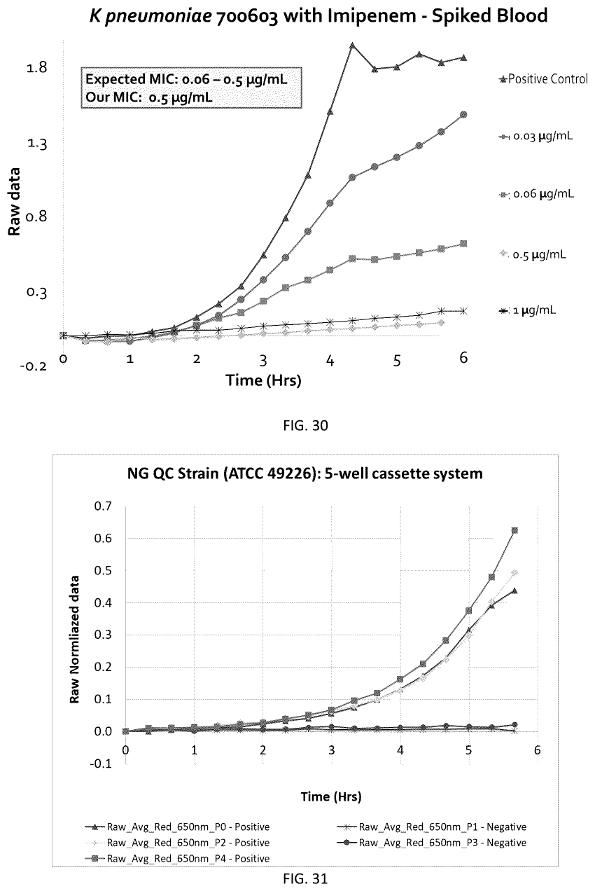

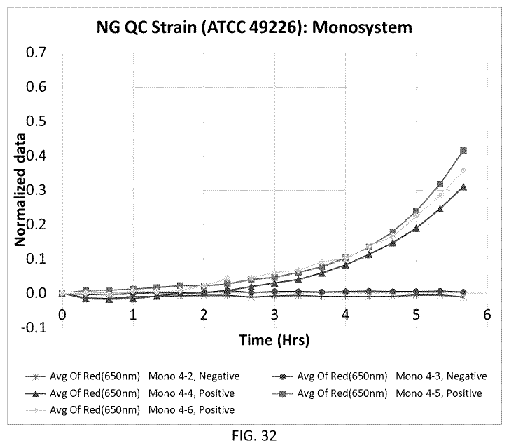

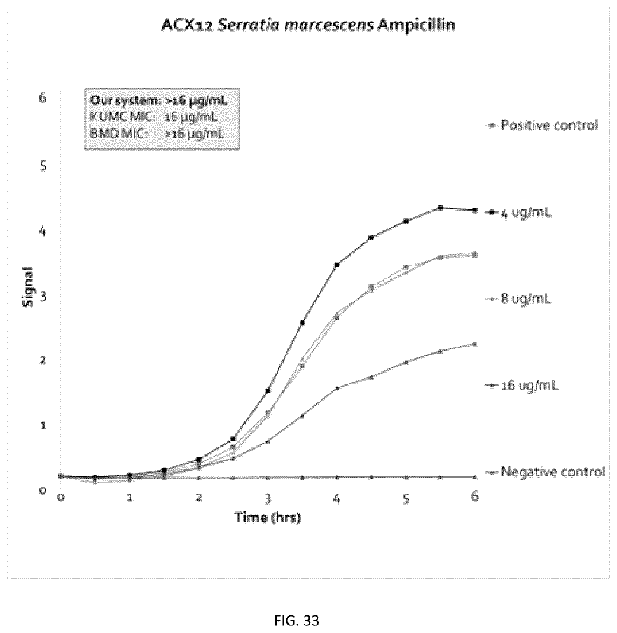

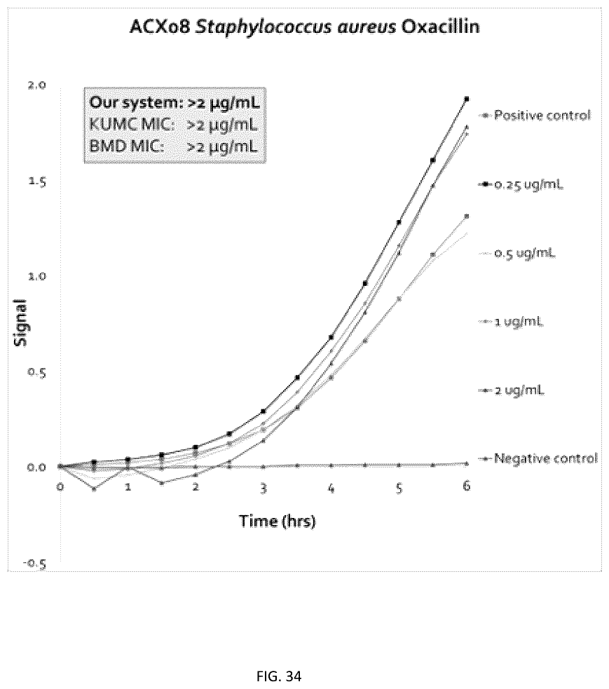

Normalization formula: (Readingₓ−Reading₀)/Reading₀
FIG. 21: 5well-V1; NG serial dilution—non-normalized;
   This figure illustrates the non-normalized (raw data) values from our device (before any data processing) vs the N. gonorrhoeae in our medium with OD values between 0-0.1 (similar to McF) separated out by wavelengths (450 nm to 650 nm).
FIG. 22: 650 nm;
   This figure illustrates the Normalized reading (of 650 nm wavelength output) from our instrument over time for NG AR 0165 (Neisseria gonorrhoeae antibiotic resistant isolate 0165) tested against antibiotic Ciprofloxacin (a fluoroquinolone) with expected MIC value (per CDC agar dilution testing) of 8 ug/mL;
   Our MIC based on the curves is: 4 ug/mL and is within +/− one doubling dilution of the expected MIC values;
   Thereby this test result is thereby in agreement with the expected value.
FIG. 23: 650 nm;
   This figure illustrates the Normalized reading (650 nm wavelength output results) from the instrument over time for NG AR 0179 (Neisseria gonorrhoeae antibiotic resistant isolate 0179) tested against antibiotic Tetracycline with expected MIC value (per CDC agar dilution testing) of 1 ug/mL;
   Our MIC based on the curves is: 2 ug/mL and is within +/− one doubling dilution of the expected MIC values;
   Thereby this test result is thereby in agreement with the expected value.
FIG. 24: This figure illustrates the Normalized reading (650 nm wavelength output results) from the instrument over time for NG 49226 (QC strain) tested against antibiotic Ciprofloxacin with expected MIC value range (per CDC agar dilution testing) of 0.001-0.008 ug/mL; Our MIC based on the curves is: 0.002 ug/mL and is within expected MIC range and is thereby in agreement with the expected range.
FIG. 25: This figure illustrates the Normalized reading (650 nm wavelength output results) from the instrument over time for E coli 25922 (QC strain) tested against antibiotic Ciprofloxacin with expected MIC value range (BMD method) of 0.004-0.016 ug/mL; Our MIC based on the curves is: 0.008 ug/mL and is within expected MIC range and is thereby in agreement with the expected range.
   Note: This table demonstrates and confirms that other organisms can be used with this system
FIG. 26: This figure illustrates the Normalized reading from the instrument over time for Neisseria gonorrhoeae 49226 (QC strain) tested against antibiotic Cefixime with expected MIC value of 0.004-0.03 ug/mL; Our MIC based on the curves is: 0.008 ug/mL and is within expected MIC range and is thereby in agreement with the expected range.
FIG. 27: This figure illustrates the Normalized reading from the instrument over time for a AR Isolate AR 0817 tested against antibiotic Cefixime with expected MIC value of 0.125 ug/mL.
   Our MIC from the instrument is determined to be: 0.125 ug/mL and is matched the expected MIC value.
FIG. 28: This figure illustrates the Normalized reading from the instrument over time for S aureus 29213 (QC strain) in cation adjusted Mueller Hinton Broth (CAMHB) tested against antibiotic Tetracycline with expected MIC value of 0.12-1 ug/mL;
   Our MIC based on the curves is: 0.008 ug/mL and is within expected MIC range and is thereby in agreement with the expected range.
FIG. 29: This figure illustrates the Normalized reading from the instrument over time for E faecalis 29212 (QC strain) in CAMHB with blood (to mimic a positive blood culture vial) tested against antibiotic Vancomycin with expected MIC value of 1-4 ug/mL;
   Our MIC based on the curves is: 4 ug/mL and is within expected MIC range and is thereby in agreement with the expected range.
FIG. 30: This figure illustrates the Normalized reading from the instrument over time for K pneumoniae 700603 in CAMHB with blood (to mimic a positive blood culture vial) tested against antibiotic Imipenem with expected MIC value of 0.06-0.5 ug/mL;
   Our MIC based on the curves is: 0.5 ug/mL and is within expected MIC range and is thereby in agreement with the expected range. This also demonstrates the time to results as early as 2.5-3 hours for this sample.
FIG. 31: This figure illustrates the Normalized reading from the 5-well prototype system over time of our proprietary media with and without the bacteria Neisseria gonorrhoeae, demonstrating rapid time to detection of the presence of pathogen in samples.
FIG. 32: This figure illustrates the Normalized reading from the mono-well prototype system over time of our proprietary media with and without the bacteria Neisseria gonorrhoeae. Comparison of 5-well to the mono-well prototype results indicate the improved signal from the 5-well system based on the many improvements described in the document.
FIG. 33: This figure illustrates the Normalized reading from the instrument over time of Serratia marcescens in diluted positive blood culture sample obtained from the clinical lab and tested against antibiotic ampicillin across different wells with the expected MIC value (based on in-house broth microdilution test) of >16 ug/mL.
   Our MIC based on the curves is: 16 ug/mL and is within expected MIC range and is thereby in agreement with the expected range. This also demonstrates the time to results as early as 2.5-3 hours for this sample.
FIG. 34: This figure illustrates the Normalized reading from the instrument over time of Staphylococcus aureus in diluted positive blood culture sample obtained from the clinical lab and tested against antibiotic Oxacillin across different wells with the expected MIC value (based on in-house broth microdilution test) of >2 ug/mL.
   Our MIC based on the curves is: >2 ug/mL and is within expected MIC range and is thereby in agreement with the expected range. This also demonstrates the rapid time to results as early as 3 hours for this sample.

Any method described herein may incorporate any design element contained within this application and any other document/application incorporated by reference herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention may be embodied in other forms without departing from the spirit and the essential attributes thereof, and, accordingly, reference should be made to the appended claims, rather than to the foregoing specification, as indicating the scope of the invention. The invention illustratively disclosed herein suitably may be practiced in the absence of any element which is not specifically disclosed herein.

1. A system for culturing and, or detecting analytes, or antimicrobial susceptibility testing comprising:
a reader which includes:
one or more processors, a computer readable memory, and a computer readable storage medium operatively associated with a computing device;
one or more cassette slots designed to accept a cassette and operatively associated with said computing device;
one or more circuit boards operatively associated with each cassette slot and said computing device, each circuit board including:
one or more light sources secured to the circuit board;
one or more light sensors secured to the circuit board;
programming instructions to facilitate the growth of one or more samples/specimen within the reader;
programming instructions to operate the light sources on the circuit board;
programming instructions to operate the light sensors on the circuit board;
programming instructions to record data generated by the light sensors;
programming instructions to interpret the data generated by light sensors;
programming instructions to display the data generated by the light sensors;
one or more cassettes operatively associated with the one or more cassette slots, each cassette including:
a housing to secure cassette components, the housing including an external shell and an internal surface;
one or more well carrier channels within the housing; and
a plurality of apertures on the external shell through to the internal shell;
one or more well carriers which will be housed in the one or more cassettes, each well carrier including:
a feed line including an outer surface and a lumen;
a port secured to one end of the feed line; and
one or more reaction wells operatively associated with the feed line and
open to the lumen of the feed line.

2. The system of claim 1 wherein the housing of each cassette is a dual clam-shell design.

3. The system of claim 1 wherein each well carrier further includes one or more venting ports to allow gas to vent as the well carrier is filled with a liquid sample.

4. The system of claim 1 wherein each reaction well further includes one or more mixing beads.

5. The system of claim 1 wherein each well carrier further includes one or more venting membranes.

6. The system of claim 1 wherein each reaction well further includes one or more venting membranes.

7. The system of claim 1 further comprising one or more internal temperature sensors located within the cassette.

8. The system of claim 1 further comprising one or more mirrors/reflectors operationally associated with each reaction well to aid in directing the light source through each reaction well and to the light sensor.

9. The system of claim 1 wherein one or more of the cassette slots is climate controlled.

10. The system of claim 1 further comprising a growth medium within the plurality of reaction wells.

11. A method for testing a sample comprising the steps of:
(a) providing a system for conducting antimicrobial susceptibility testing comprising:
a reader which includes:
one or more processors, a computer readable memory, and a computer readable storage medium operatively associated with a computing device;
one or more cassette slots designed to accept a cassette and operatively associated with said computing device;
one or more circuit boards operatively associated with each cassette slot and said computing device, each circuit board including:
one or more light sources secured to the circuit board;
one or more light sensors secured to the circuit board;
programming instructions to facilitate the growth of one or more samples/specimen within the reader;
programming instructions to operate the light sources on the circuit board;
programming instructions to operate the light sensors on the circuit board;
programming instructions to record data generated by the light sensors;
programming instructions to interpret the data generated by light sensors;
programming instructions to display the data generated by the light sensors;
one or more cassettes operatively associated with the one or more cassette slots, each cassette including:
a housing to secure cassette components, the housing including an external shell and an internal surface;
one or more well carrier channels within the housing; and
a plurality of apertures on the external shell through to the internal shell;
one or more well carriers which will be housed in the one or more cassettes, each well carrier including:
a feed line including an outer surface and a lumen;
a port secured to one end of the feed line; and
one or more reaction wells operatively associated with the feed line and open to the lumen of the feed line;
(b) providing a growth medium within each reaction well;
(c) providing a delivery device which is used to introduce samples/specimen into the system, the delivery device containing a sample/specimen;
(d) injecting the sample/specimen into each port, which flows into the feed line associated with each port, which then flows into each of the plurality of reaction wells associated with each feed line;
(e) inserting the cassette into the cassette slot within the reader;
(f) mixing the sample/specimen within each reaction vessel;
(g) activating the programming instructions to facilitate the growth of the sample/specimen within the reader;
(h) activating the programming instructions to operate the light sources on the circuit board;
(i) activating the programming instructions to operate the light sensors on the circuit board;
(j) activating the programming instructions to record data generated by the light sensors;
(k) activating the programming instructions to interpret the data generated by light sensors; and
(l) activating the programming instructions to display the data generated by the light sensors.

12. The method of claim 11 wherein the housing of each cassette is a dual clam-shell design.

13. The method of claim 11 wherein each well carrier further includes one or more venting ports to allow gas to vent as the well carrier is filled with a liquid sample.

14. The method of claim 11 wherein each reaction well further includes one or more mixing beads.

15. The method of claim 11 wherein each well carrier further includes one or more venting membranes.

16. The method of claim 11 wherein each reaction well further includes one or more venting membranes.

17. The method of claim 11 further comprising one or more internal temperature sensors located within the cassette.

18. The method of claim 11 further comprising one or more mirrors/reflectors operationally associated with each reaction well to aid in directing the light source through each reaction well and to the light sensor.

19. The method of claim 38 wherein one or more of the cassette slots is climate controlled.

20. The method of claim 38 further comprising a growth medium within the plurality of reaction wells.
